(19) United States
(12) Patent Application Publication
(10) Pub. No.: US 2025/0111898 A1
(43) Pub. Date: Apr. 3, 2025

(54) TRACKING AND MODIFYING CLUSTER LOCATION ON NUCLEOTIDE-SAMPLE SLIDES IN REAL TIME

(71) Applicant: Illumina, Inc., San Diego, CA (US)

(72) Inventors: Gavin Derek Parnaby, San Diego, CA (US); Eric Jon Ojard, San Diego, CA (US); Rami Mehio, San Diego, CA (US)

(21) Appl. No.: 18/900,557

(22) Filed: Sep. 27, 2024

Related U.S. Application Data
(60) Provisional application No. 63/587,001, filed on Sep. 29, 2023.

(51) Int. Cl. G16B 30/00; G16B 40/10

(52) U.S. Cl. CPC G16B 30/00; G16B 40/10

(57) ABSTRACT

This disclosure describes embodiments of methods, systems, and non-transitory computer readable media that can (i) estimate a location error for a predicted location of a cluster of oligonucleotides based on the cluster's signal and (ii) modify the predicted location of the cluster to improve signal detection and base calling on a sequencing device. For example, the disclosed systems can receive a signal from a cluster of oligonucleotides at a predicted location. The disclosed systems can further determine an intensity-value error between an intensity value and an expected intensity value for the signal at the predicted location. Based on the intensity-value error and intensity values from other locations (e.g., other clusters of oligonucleotides) within the region, the disclosed system can determine an estimated location error for the predicted location. The disclosed systems can modify the predicted location of the cluster of oligonucleotides based on the estimated location error.

### CROSS-REFERENCE TO RELATED APPLICATIONS

This application claims priority to and the benefit of U.S. Provisional Patent Application No. 63/587,001, entitled “TRACKING AND MODIFYING CLUSTER LOCATION ON NUCLEOTIDE-SAMPLE SLIDES IN REAL TIME,” filed on Sep. 29, 2023, which is incorporated herein by reference in its entirety.

### BACKGROUND

In recent years, biotechnology firms and research institutions have improved hardware and software for sequencing nucleotides and determining nucleobase calls for genomic samples. For instance, some existing sequencing machines and sequencing-data-analysis software (together “existing sequencing systems”) predict individual nucleobases within sequences by using conventional Sanger sequencing or sequencing-by-synthesis (SBS) methods. When using SBS, existing sequencing systems can monitor millions to billions of oligonucleotides being synthesized in parallel and in clusters from templates to predict nucleobase calls for growing nucleotide reads. A camera in many existing sequencing systems captures images of irradiated fluorescent tags incorporated into oligonucleotides or images of other signals indicating incorporated nucleobases. To effectively identify which signals come from which clusters of oligonucleotides in a sequencing cycle—and determine accurate base calls for such clusters-existing systems estimate locations of the clusters of oligonucleotides on a flow cell or other nucleotide-sample slide. After capturing such images, some existing sequencing systems determine nucleobase calls for nucleotide reads corresponding to the oligonucleotides. By iteratively incorporating nucleotide bases into the oligonucleotides and capturing images of emitted light signals (or other signals) in various sequencing cycles, existing sequencing systems can determine the sequence of nucleotide bases present in the genomic samples.

To facilitate cluster-location tracking, some existing sequencing systems estimate the location of clusters of oligonucleotides during a registration process for a camera by (i) identifying fiducials or other physical markers embedded on nucleotide-sample slides, (ii) interpolating between such markers, and (iii) further compensating for optical aberrations. An equalizer in such existing sequencing systems works best when it can center a signal from a cluster on the center location of the cluster to adequately or optimally compensate for a point spread function (PSF) and noise in the environment. If, however, the existing sequencing system's equalizer is not centered on a cluster's location, the equalizer cannot optimize or otherwise adjust/customize the response (e.g., PSF) of the signal from the cluster. In particular, when an equalizer has not correctly centered on a cluster's location, existing sequencing systems cannot minimize the mean-squared-average-error across multiple signals from clusters on the flow cell. This sub-optimal process causes an existing sequencing system's equalizer to incorrectly account for noise that varies across signals from clusters and reduces the equalizer's ability to optimize the response by minimizing the overall mean-squared-average error. Moreover, such inadequacies consequently, introduce errors in the process of determining base calls from the SNR-adjusted signal from a cluster.

Despite recent advances to cluster-location estimates and equalizers, existing sequencing systems sometimes inaccurately estimate the locations of clusters of oligonucleotides on flow cells or other nucleotide-sample slides, rely on incorrect locations of clusters, and, consequently, determine incorrect base calls. While fiducials and other physical markers help as reference points for cluster location within an image, such physical markers on a nucleotide-sample slide do not support accurately accounting for image jitter—that is, random movement of image lines due to corruption of synchronization signals. Nor can fiducials shown in an image help account for variation of cluster locations within the nucleotide-sample slide (e.g., clusters located near edges of an image region versus a center of an image region) or a thermal expansion of the nucleotide-sample slide-both of which make estimating the location of an individual cluster more difficult. Because existing sequencing systems cannot accommodate jitter, cluster-location variation, and/or thermal expansion, such existing systems sometimes do not accurately identify the signal emitted from the cluster of oligonucleotides and captured by an image. Without a better model to compensate for such location-tracking issues, such existing sequencing systems sometimes do not align spatial dispersion compensation (e.g., as performed by an equalizer) appropriately (or optimally) with respect to every cluster (or corresponding well) within a region of the nucleotide-sample slide. This failure to align spatial dispersion compensation leads to a reduced signal-to-noise ratio (SNR) for the signal. Not only can incorrect cluster-location estimates cause incorrect base calls from poorly SNR-adjusted signals, but existing sequencing systems exhibiting such cluster-location-estimate inaccuracies tend to produce fewer clusters of oligonucleotides that pass quality filters and/or clusters with fewer portions or subsequences of nucleotide reads that can be used for mapping and aligning (e.g., because of hard or soft clipping).

To compensate for cluster-location-estimate inaccuracies, some existing sequencing systems have used (or attempted to use) nucleotide-sample slides with a denser distribution of fiducials or other physical markers. But adding fiducials introduces both space and computing constraints in a technical environment in which the sequencing speed of a sequencing device is critical. Additional fiducials or other markers consume space on the substrate of a nucleotide-sample slide and leave less space for wells and/or clusters of oligonucleotides. Consequently, existing sequencing systems with increased fiducial densities reduce the number of clusters that can be sequenced in a sequencing run and reduce throughput in a microscopic space that supports millions to billions of clusters. Exasperating the space constraints, additional computational processing during registration is required to process and calibrate as the number of fiducials or other physical markers increase in density on a nucleotide-sample slide. Further, as other technological improvements increase the speed of sequencing cycles and corresponding sequencing runs, the increased levels of mechanical movement will only compound the thermal expansion of the nucleotide-sample slide and thereby reduce the utility and reliability of physical fiducials on an expanding substrate.

These, along with additional problems and issues exist in existing sequencing systems.

### BRIEF SUMMARY

This disclosure describes implementations of methods, non-transitory computer-readable media, and systems that can solve one or more of the foregoing (or other problems) in the art. To solve such problems, the disclosed systems can (i) estimate a location error for a predicted location of a cluster of oligonucleotides based on the cluster's signal and (ii) modify the predicted location of the cluster to improve signal detection and base calling on a sequencing device. For example, the disclosed systems can receive a signal from a cluster of oligonucleotides at a predicted location. The disclosed systems can further determine an intensity-value error between an intensity value and an expected intensity value for the signal at the predicted location. Based on the intensity-value error and intensity values from other locations (e.g., other clusters of oligonucleotides) within the region, the disclosed system can determine an estimated location error for the predicted location. The disclosed systems can modify the predicted location of the cluster of oligonucleotides based on the estimated location error.

The disclosed systems can utilize the estimated location error associated with clusters of oligonucleotides for a variety of base-calling applications described further below. By improving a predicted location of a cluster of oligonucleotides in a given sequencing cycle, for example, the disclosed systems can more accurately locate clusters of oligonucleotides, more accurately adjust a signal for a cluster based on improved location, and determine improved nucleobase calls for the cluster based on better adjusted signals.

Additional features and advantages of one or more implementations of the present disclosure are outlined in the description which follows, and in part will be obvious from the description, or may be learned by the practice of such example implementations.

### BRIEF DESCRIPTION OF THE DRAWINGS

The detailed description provides one or more implementations with additional specificity and detail through the use of the accompanying drawings, as briefly described below.

FIG. 1 illustrates a diagram of an environment in which a location-error-prediction system can operate in accordance with one or more implementations.

FIG. 2 illustrates an overview of the location-error-prediction system determining an estimated location error for a predicted location of the cluster of oligonucleotides and modifying the predicted location of the cluster of oligonucleotides based on the estimated location error in accordance with one or more implementations.

FIGS. 3A-3C illustrate diagrams of sample clusters of oligonucleotides with either incorrect or correct predicted locations on a nucleotide-sample slide in accordance with one or more implementations.

FIG. 4 illustrates location-error-prediction system iteratively modifying a predicted location of a cluster of oligonucleotides to more closely align with a corrected location of the cluster of oligonucleotides in accordance with one or more implementations.

FIG. 5 illustrates the location-error-prediction system modifying the predicted location of the cluster of oligonucleotides based on the estimated location error in accordance with one or more implementations.

FIG. 6 illustrates improved cluster location tracking by utilizing the disclosed location-error-prediction system in accordance with one or more implementations.

FIG. 7 illustrates a flowchart of a series of acts for modifying the predicted location of a cluster of oligonucleotides based on the estimated location error in accordance with one or more implementations.

FIG. 8 illustrates a block diagram of an example computing device for implementing one or more implementations of the present disclosure.

### DETAILED DESCRIPTION

This disclosure describes one or more implementations of a location-error-prediction system that can determine an estimated location error for a predicted location of a cluster of oligonucleotides based on the cluster's signal and modify the predicted location of the cluster of oligonucleotides based on the estimated location error. For example, the location-error-prediction system receives a signal from a cluster of oligonucleotides at a predicted location for the cluster of oligonucleotides within a region of a nucleotide-sample slide and determines an intensity-value error between an intensity value for the signal and an expected intensity value for the signal at the predicted location within the region. The location-error-prediction system further determines an estimated location error for the predicted location based on the intensity-value error and intensity values of other locations (e.g., cluster of oligonucleotides) within the region. Based on the estimated location error, the location-error-prediction system modifies the predicted location of the cluster of oligonucleotides.

As suggested above, in one or more embodiments, the location-error-prediction system receives a signal from a cluster of oligonucleotides at a predicted location within a region of a nucleotide-sample slide. For example, the location-error-prediction system can capture an image of the signal at the predicted location and extract/detect intensity values (e.g., wavelengths and/or brightness values) for the signal emitted by a cluster of oligonucleotides at the predicted location during a sequencing cycle. In some cases, the location-error-prediction system can receive signals from other clusters of oligonucleotides within the same region of the nucleotide-sample slide. In certain embodiments, the location-error-prediction system then utilizes the signals emitted from other clusters of oligonucleotides within the region to determine the estimated location error for the cluster of oligonucleotides.

From the received signals, the location-error-prediction system can determine an intensity-value error between the received and expected intensity values at the predicted location. In particular, the location-error-prediction system determines the intensity-value error between an intensity value (e.g., measured intensity value) and expected intensity value for the signal at the predicted location within the region of the nucleotide-sample slide. For example, the location-error-prediction system determines the intensity value for the signal at the predicted location by measuring a wavelength and/or brightness of the signal at the predicted location. In some embodiments, the location-error-prediction system determines the expected intensity value for the signal based on the illumination status (e.g., ON/OFF or continuous/dynamic illumination indicator) of the signal within a given channel. In particular, the location-error-prediction system can determine the expected intensity value for the signal by utilizing an average (e.g., centroid) of intensity values associated with intensity-value boundaries (e.g., nucleotide clouds) of a certain base (e.g., A, C, G, or T) within the given channel.

Based on the intensity-value error and intensity values from other locations (e.g., nearby clusters of oligonucleotides or pixels) within the region, the location-error-prediction system can determine an estimated location error for the predicted location of the cluster of oligonucleotides. The estimated location error indicates the degree and/or direction of misalignment between the predicted location of the cluster of oligonucleotides and the modified location of the cluster of oligonucleotides. In one or more implementations, the location-error-prediction system determines the estimated location error by (i) combining (e.g., subtracting) the intensity-value error with the intensity values of other clusters of oligonucleotides adjacent to the cluster of oligonucleotides or (ii) combining the intensity-value error with intensity values of pixels positioned adjected to the predicted location of the cluster of oligonucleotides. In some embodiments, the location-error-prediction system can determine an average estimated location error for clusters of oligonucleotides within the region. Additionally, or alternatively, in certain implementations, the location-error-prediction system can determine the estimated location error for multiple dimensions, multiple channels, and/or multiple sequencing cycles.

Having determined the estimated location error, the location-error-prediction system can utilize the estimated location error to modify the location of the predicted location to more closely align with the actual location of the cluster of oligonucleotides. In some embodiments, the actual location of the cluster of oligonucleotides can be the center of a group of oligonucleotides. In some embodiments, the actual location of the cluster of oligonucleotides can be the center of the well holding the cluster of oligonucleotides. As mentioned above, the estimated location error indicates the degree and direction of misalignment between the predicted location of the cluster of oligonucleotides and the modified location of the cluster of oligonucleotides. Based on the estimated location error, the location-error-prediction system can modify the predicted location by moving the predicted location in a direction opposite from the direction indicated by the estimated location error.

In some embodiments, for instance, the location-error-prediction system modifies the predicted locations for clusters of oligonucleotides within a region based on the average of the estimated location errors for the region within the nucleotide-sample slide. For example, if the average estimate location error indicates that the predicted locations for the clusters of oligonucleotides within the region are left of the actual location of the clusters of oligonucleotides within the region, the location-error-prediction system can modify the predicted locations of the clusters of oligonucleotides by moving them to the right. In certain implementations, the location-error-prediction system modifies the predicted locations of clusters of oligonucleotides based on the average estimated location error for a number of cycles. In certain cases, the location-error-prediction system can modify the predicted location of a single cluster of oligonucleotides, row of cluster of oligonucleotides, and/or column of cluster of oligonucleotides. As described further below, the location-error-prediction system can likewise modify the predicted location of one or more clusters in two or more dimensions independently (e.g., one location-prediction modification in a horizontal dimension and another location-prediction modification in a vertical dimension or multiple location-prediction modifications to adjust for clusters in a hexagonal layout).

The location-error-prediction system provides several advantages over existing sequencing systems. For instance, the location-error-prediction system improves the accuracy of cluster location tracking by identifying an improved sampling location (e.g., by an equalizer) for a cluster of oligonucleotides. As noted above, the location-error-prediction system can (i) estimate a location error for a predicted location of a cluster of oligonucleotides based on the cluster's (and/or other clusters') signal and (ii) modify the predicted location of the cluster to improve signal detection and base calling on a sequencing device. By performing (i) and (ii), the location-error-prediction system aligns a signal of a cluster with the center of the cluster, where interference from other clusters is relatively closer to zero than other positions. Because the location-error-prediction system samples the cluster of oligonucleotides at a more accurate predicted location—where interference from other clusters is lower than at other predicted locations—the location-error-prediction system accurately measures the intensity values of clusters. With an improved predicted location of a target cluster of oligonucleotides in a given sequencing cycle, the location-error-prediction system can better compensate for a point spread function (PSF) using an equalizer, more accurately estimate the intensity value for each cluster, and based on the improved intensity value more accurately estimate a signal-to-noise ratio (SNR) for the signal. By better compensating for the PSF and improving the estimated SNR, the location-error-prediction system 106 generates a more accurate adjusted signal upon which to determine a more accurate base call. By further performing (i) and (ii) during or across sequencing cycles, in some embodiments, the location-error-prediction system can improve the sampling location for the cluster of oligonucleotides in real time during a sequencing run on a sequencing device. Consequently, the location-error-prediction system can improve the sampling location between cycles or after a subset of cycles and can adjust the predicted location of either a specific cluster or multiple clusters within a region on the nucleotide-sample slide based on an average location error for the region.

In addition to improved accuracy, the location-error-prediction system improves the space and computing efficiency relative to alternative approaches to cluster-location tracking and adjustment. As indicated above, some existing sequencing system attempt to use nucleotide-sample slides with a denser distribution of fiducials (or other physical markers) to better estimate cluster location using the fiducials as references. But such a denser fiducial distribution consumes space that could be used for additional clusters and requires further computation based on additional fiducials to estimate cluster locations during registration cycles. In contrast to a problematic increase in fiducial density, the location-error-prediction system improves cluster-location prediction by (i) estimating a location error for a predicted location of a cluster of oligonucleotides based on the cluster's (and/or other clusters') signal and (ii) modifying the predicted location of the cluster. By doing so in a given sequencing cycle, the location-error-prediction system can accurately and dynamically track the location of clusters of oligonucleotides while maintaining, eliminating, or reducing the number of fiducials on the nucleotide-sample slide, resulting in higher sequencing throughput and lower computation costs. In some embodiments, as described below, the location-error-prediction system utilizes a location-error-detection loop that efficiently updates the predicted locations of clusters. If fiducials exist on the nucleotide-sample slide, the location-error-prediction system can conserve computational processing by registering fewer fiducials to initialize a sequencing device than the denser fiducials approach described above.

Beyond improved efficiency, the location-error-prediction system can facilitate increased sequencing speeds on a specialized sequencing device relative to existing sequencing systems. For instance, as sequencing speeds increase, systems need to tolerate greater levels of mechanical movement. Such movement results in greater degrees of misalignment between an encoder and the center of the cluster. As described above and below, the location-error-prediction system can accurately track/identify the locations of clusters by determining and removing the estimated location error-without adding a denser distribution of fiducials that would decrease the number of clusters on a nucleotide-sample slide and consequently sequencing throughput. In particular, the location-error-prediction system can track the location of clusters and compensate for misalignments/location errors during a sequencing cycle or a sequencing run based on the clusters' signals.

As illustrated by the foregoing discussion, the present disclosure utilizes a variety of terms to describe features and advantages of the location-error-prediction system. As used herein, the term “cluster of oligonucleotides” (or simply “cluster”) refers to a localized group or collection of DNA or RNA molecules on a nucleotide-sample slide, such as a flow cell, or other solid surface. In particular, a cluster includes tens, hundreds, thousands, or more copies of a cloned or the same DNA or RNA segment. For example, in one or more embodiments, a cluster includes a grouping of oligonucleotides immobilized in a section of a flow cell or other sample slide. In some embodiments, clusters are evenly spaced or organized in a systematic structure within a patterned flow cell. By contrast, in some cases, clusters are randomly organized within a non-patterned flow cell. A cluster of oligonucleotides can be imaged utilizing one or more light signals. For instance, an oligonucleotide-cluster image may be captured by a camera during a sequencing cycle of light emitted by irradiated fluorescent tags incorporated into oligonucleotides from one or more clusters on a flow cell.

Further, as used herein the term “signal” refers to a signal emitted, reflected, or otherwise communicated from a labeled nucleotide base or a group of labeled nucleotide bases (e.g., labeled nucleotide bases added to a cluster of oligonucleotides). In particular, a signal can refer to a signal indicating the type of base. For example, a signal can include a light signal emitted or reflected from a fluorescent tag of a nucleotide base or fluorescent tags of multiple nucleotide bases incorporated into oligonucleotides. In some embodiments, the signal can include a voltage emitted by the cluster of oligonucleotides indicating the base. As indicated above, a nucleobase incorporated into a cluster may (in response to a laser) likewise emit a signal that can be identified as a mixture of dyes (or a mixture of fluorescent tags) that together indicate the nucleobase type. In some implementations, the location-error-prediction system triggers the signal through an external stimulus, such as a laser or other light source. In some cases, the location-error-prediction system triggers the signal through some internal stimuli. Further, in some embodiments, the location-error-prediction system observes the signal using a filter applied when capturing an image of the nucleotide-sample slide (e.g., region of the nucleotide-sample slide).

As used herein, the term “nucleotide-sample slide” (or “nucleotide-sample substrate”) refers to a plate or substrate, such as a flow cell, comprising oligonucleotides for sequencing nucleotide sequences from genomic samples or other sample nucleic-acid polymers. In particular, a flow cell can refer to a substrate containing fluidic channels through which reagents and buffers can travel as part of sequencing. For example, in one or more embodiments, the flow cell (e.g., a patterned flow cell or non-patterned flow cell) may comprise small fluidic channels and oligonucleotide samples that can be bound to adapter sequences on the substrate. In other implementations, a flow cell can be an open substrate with one or more regions for oligonucleotide samples to be analyzed and the oligonucleotide samples may be positioned using charged pads or other means. In yet another implementation, the nucleotide-sample substrate can be a membrane having a nanopore through which one or more oligonucleotide samples may pass. As indicated above, a flow cell can include tiles and wells (e.g., nanowells) comprising clusters of oligonucleotides. In some cases, a patterned flow cell may take on, but is not limited to, a square, hexagonal, and/or diamond shape. A nucleotide-sample slide may also include fiducials.

Relatedly, as used herein, the term “fiducial” refers to reference point or physical marker on the nucleotide-sample slide. In particular, a fiducial includes a physical marking that is etched on or embedded into a surface of a nucleotide-sample slide. As noted above, in some cases, fiducials help align an imaging system with regions of a nucleotide-sample slide and assists in identifying the location of clusters on the nucleotide-sample slide.

Relatedly, as used herein, the term “region of a nucleotide-sample slide” (or “nucleotide-sample slide region”) refers to an area that is part of a nucleotide-sample slide. In particular, a region of a nucleotide-sample slide can refer to a discrete portion of a nucleotide-sample slide that differs from other portions of the nucleotide-sample slide. For instance, a region of a nucleotide-sample slide can include a subsection of patterned flow cell comprising one or more wells (e.g., a nano-wells) or a discrete subsection of a non-pattered flow cell (e.g., a subsection corresponding to one or more clusters). In some cases, a region (e.g., section) of a nucleotide-sample slide includes a tile or a sub-tile of a flow cell having clusters of oligonucleotides growing in parallel.

As used herein, the term “channel” refers to a range or filter of light, intensity, or color used to detect and/or measure a signal from a cluster of oligonucleotides. For example, a channel can include a particular range of light, intensity, or color of a laser used to illicit a fluorescent signal from fluorescent tags on nucleobases incorporated into oligonucleotides within a cluster. In some embodiments, the location-error-prediction system utilizes a two-channel implementation by, for instance, using two different ranges of light, intensities, or colors to illicit signals from clusters per sequencing cycle and capturing two corresponding images of a region of a nucleotide-sample slide per sequencing cycle. The first and second images can capture the intensity values of the emitted signal from the clusters that correspond to first and second light ranges. In some embodiments, the location-error-prediction system can utilize a single channel implementation, three-channel implementation, or four-channel implementation.

Further, the term “predicted location” refers to an approximated position of a cluster of oligonucleotides on a nucleotide-sample slide. For instance, the location-error-prediction system can receive an image of a signal of a cluster of oligonucleotides and, based on pixels the image showing a response indicative of incorporated nucleobases, determine a predicted location of the cluster. For example, in some embodiments, the location-error-prediction system can determine the predicted location of the cluster by processing the image of the signal and utilizing location algorithms on the processed image.

As used herein, the term “intensity value” refers to a value indicating a characteristic or attribute of a signal emitted, reflected, or otherwise communicated from a labeled nucleotide base or a group of labeled nucleotide bases from a cluster of oligonucleotides. In particular, an intensity value can refer to a value associated with a color intensity (e.g., wavelength) or a light intensity (e.g., brightness). In some cases, the location-error-prediction system captures several images of a cluster of oligonucleotides with labeled nucleotide bases using different channels. Thus, an intensity value of a signal can correspond to the intensity of the signal as observed through a particular channel. In one or more embodiments, the intensity value is a measured degree of intensity for a cluster of oligonucleotides at the predicted location, and the location-error-prediction system can accordingly be applied to 16 quadrature amplitude modulation (QAM) modulation or pulse amplitude modulation (PAM) 4 modulation (e.g., using amplitude to encode base-call information).

Additionally, as used herein, the term “expected intensity value” refers to a value indicating an illumination state of a signal associated with a cluster of oligonucleotides (e.g., ON/OFF). In particular, an expected intensity value includes an expected value indicating an illumination state (e.g., ON/OFF) by a particular nucleotide base (A, C, G, T) in a particular channel. For instance, in some cases, the expected intensity value refers to an average of (or centroid for) intensity values associated with the ON/OFF status of a particular channel. In certain implementations, the expected intensity value is an average of intensity values falling within the intensity-value boundaries (e.g., nucleotide clouds) of a certain base (A, C, G, or T). In certain implementations, the centroid value of the intensity channels is based on the intensity values of one or more sequencing cycles. In some embodiments, the expected intensity value is the same for all cluster of oligonucleotides within the region.

As used herein, the term “intensity-value error” refers to the difference between a detected or measured intensity value of a cluster of oligonucleotides and an expected intensity value of the cluster of oligonucleotides. For instance, the intensity-value error can include the difference between a measured intensity value of a cluster of oligonucleotides for a particular channel and an expected intensity value given an illumination status (e.g., ON/OFF) of the cluster of oligonucleotides in the particular channel.

As used herein, the term “illumination indicator” refers to an indicator of whether a cluster of oligonucleotides is illuminated by or emits an intensity of light in a particular frequency band during a sequencing cycle. In particular, an illumination indicator represents whether (or a degree to which) a cluster of oligonucleotides (i) comprises labeled nucleotides emitting a particular intensity of light in a particular frequency (e.g., frequency band) to become illuminated (e.g., on or active) or (ii) does not comprise labeled nucleotide bases such that it is not illuminated (e.g., off, undetectable, or inactive) by a particular intensity of light in a particular frequency (e.g., frequency band) in an intensity channel during sequencing. In some cases, an illumination indicator can take a couplet format. For example, if a cluster of oligonucleotides incorporates nucleobases with fluorescent tags or other labels that (in response to a light or laser) illuminate or emit a light intensity in a particular frequency (e.g., frequency band) of light in a channel during a sequencing cycle, the “on” or “illuminated” status for an illumination indicator can be represented by a one. Conversely, if a cluster of oligonucleotides does not incorporate (or incorporates too few) nucleobases with fluorescent tags or other labels that (in response to a light or laser) illuminate or emit light intensity in a particular frequency in a particular frequency (e.g., frequency band) in a channel during a sequencing cycle, the “off” or “unilluminated” status for an illumination indicator can be represented by a zero. To illustrate, [1,1] can indicate that an illumination indicator for a cluster of oligonucleotides is illuminated in two different channels. While the description and figures depict illumination indicators in different channels (e.g., two channels or four channels), the location-error-prediction system can detect signals from clusters concurrently in such different channels. In some embodiments, the location-error-prediction system can identify the illumination state of the cluster within a channel by making a base call and decoding the base call to find the ON/OFF status for the cluster within each channel.

By contrast, if a polyclonal cluster of oligonucleotides incorporates nucleobases with different fluorescent tags or other labels that (in response to a light or laser) illuminate or emit light within different spectral bands in a given channel during a sequencing cycle, the status for an illumination indicator would not be entirely “on” or “off” (or not be entirely “illuminated” or “unilluminated”). In some cases, such a mixed signal from a polyclonal cluster of oligonucleotides is filtered out and discarded based on intensity-value boundaries for different types of nucleobases.

While this disclosure frequently uses illumination indicators in the form of “on” or “off” (or a corresponding “1” or “0”), an illumination indicator can be particular to a channel and is not designed to indicate a presence or absence of background noise or other light. In some implementations, an off indicator or status may indicate an undetectable signal from a cluster of oligonucleotides. Accordingly, an “off” or “0” indicator does not indicate an absence of light, but rather an estimate that a particular cluster did not incorporate (or incorporates too few) nucleobases with fluorescent tags or another label that (in response to a light or laser) illuminate or emit light intensity in a particular frequency (e.g., frequency band) in a particular channel during a sequencing cycle. Accordingly, an illumination indicator can take other formats. In the alternative to a couplet format, in some embodiments, an illumination indicator may be continuous and represent a degree to which a given cluster is illuminated during a sequencing cycle. Such a continuous illumination indicator, for example, can take the form of a metric or score (e.g., between 0 and 1) indicating a degree to which a cluster is illuminated by light emitted from a particular type of nucleotide incorporated into the cluster during a sequencing cycle.

As used herein, the term “estimated location error” refers to a measure or quantification of error between a predicted location and a corrected or modified location of the cluster of oligonucleotides. For instance, the estimated location error indicates the degree and direction of error and/or misalignment between the predicted location and corrected location of the cluster of oligonucleotides. For example, the estimated location error reflects whether the predicted location precedes or succeeds the corrected location for the cluster of oligonucleotides. In some embodiments, the location-error-prediction system determines the estimated location error by combining (e.g., subtracting or using another mathematical operation) the intensity-value error with the intensity values for other locations (e.g., other clusters of oligonucleotides) within the region. In certain implementations, the estimated location error is not a direct estimate of the distance between the predicted location of the cluster and the modified location of the cluster (e.g., location error offset). For instance, in some embodiments, estimated location error reflects the correlation between the intensity value of the signal at the predicted location of the cluster and the modified location of the cluster. For example, due to the transformation from the PSF, the estimated location error can utilize a nonlinear function to map the intensity of the signal from the cluster with the distance (e.g., location error offset) between the predicted location of the cluster and modified location of the cluster. In cases where the estimated location error represents a small location error offset, the location-error-prediction system can utilize a linear model to map the signal of the cluster to the distance between the predicted location and modified location.

As further used herein, the term “nucleotide-base call” (or simply “base call”) refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate a determination or prediction of the type of nucleobase that has been incorporated within an oligonucleotide on a nucleotide-sample slide (e.g., read-based nucleobase calls). In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). As suggested above, a single nucleobase call can be an adenine (A) call, a cytosine (C) call, a guanine (G) call, a thymine (T) call, or an uracil (U) call. In some embodiments, the type of base (e.g., adenine, cytosine, thymine, or guanine) can be determined based on intensity values for a signal emitted by labeled nucleotide bases in a cluster of oligonucleotides, such as signals in 16 quadrature amplitude modulation (QAM) or pulse amplitude modulation (PAM) 4 format.

Additionally, as used herein, the term “nucleotide-base-call data” refers to a digital file, image data, or other digital information indicating individual nucleotide bases or the sequence of nucleotide bases for a nucleic-acid polymer. In particular, nucleotide-base-call data can include intensity values (e.g., color or light intensity values for individual clusters) from images taken by a camera of a nucleotide-sample slide or other data that indicate individual nucleotide bases or the sequence of nucleotide bases for a nucleic-acid polymer. In addition, or in the alternative to intensity values, the nucleotide-base-call data may include chromatogram peaks or electrical current changes indicating individual nucleobases in a sequence. Additionally, in some embodiments, nucleotide-base-call data includes individual nucleotide-base calls identifying the individual nucleotide bases (e.g., A, T, C, or G). For example, nucleotide-base-call data can comprise data for nucleotide-base calls in a sequence for a nucleic-acid polymer, the number of nucleotide-base calls corresponding to a particular base (e.g., adenine, cytosine, thymine, or guanine), as organized in a digital file, such as a Binary Base Call (BCL) file. Further, nucleotide-base call data can include error/accuracy information, such as a quality metric associated with each nucleotide-base call. In some embodiments, nucleotide-base-call data comprises information from a sequencing device that utilizes sequencing by synthesis (SBS).

As used herein, the term “sequencing run” refers to an iterative process on a sequencing device to determine a primary structure of nucleotide sequences from a sample (e.g., genomic sample). In particular, a sequencing run includes cycles of sequencing chemistry and imaging performed by a sequencing device that incorporate nucleobases into growing oligonucleotides to determine nucleotide reads from nucleotide sequences extracted from a sample (or other sequences within a library fragment) and seeded throughout a flow cell. In some cases, a sequencing run includes replicating oligonucleotides derived or extracted from one or more genomic samples seeded in clusters throughout a flow cell. Upon completing a sequencing run, a sequencing device can generate nucleotide-base-call data in a file, such as a binary base call (BCL) sequence file or a fast-all quality (FASTQ) file.

As used herein, the term “sequencing cycle” (or “cycle”) refers to an iteration of adding or incorporating one or more nucleobases to one or more oligonucleotides representing or corresponding to sample's sequence (e.g., a genomic or transcriptomic sequence from a sample) or a corresponding adapter sequence. In some cases, a sequencing cycle includes an iteration of both incorporating nucleobases into clusters of oligonucleotides using sequencing chemistry and capturing images of such clusters attached to a nucleotide-sample slide (e.g., a flow cell). Accordingly, cycles can be repeated as part of sequencing a nucleic-acid polymer (e.g., a sample genomic sequence). For example, in one or more embodiments, each sequencing cycle involves incorporating nucleobases into either a single nucleotide read in which DNA or RNA strands are read in only a single direction or paired-end reads in which DNA or RNA strands are read from both ends but in different cycles. Further, in certain cases, each sequencing cycle involves a camera taking an image of the nucleotide-sample slide or multiple sections of the nucleotide-sample slide to generate image data for determining a particular nucleotide base added or incorporated into particular oligonucleotides. Following the image capture stage, a sequencing device can remove certain fluorescent labels from incorporated nucleotide bases and perform another sequencing cycle until the nucleic-acid polymer has been completely sequenced. In one or more embodiments, a sequencing cycle includes a cycle within an SBS run. A sequencing cycle can include one or both of an indexing cycle and a genomic sequencing cycle. For instance, one cluster of oligonucleotides or a set of clusters of oligonucleotides may be undergoing a genomic sequencing cycle in which nucleobases corresponding to a sample genomic sequence are incorporated and another cluster of oligonucleotides or another set of clusters of oligonucleotides may be concurrently undergoing an indexing cycle in which nucleobases corresponding to an indexing sequence for a nucleotide read are incorporated.

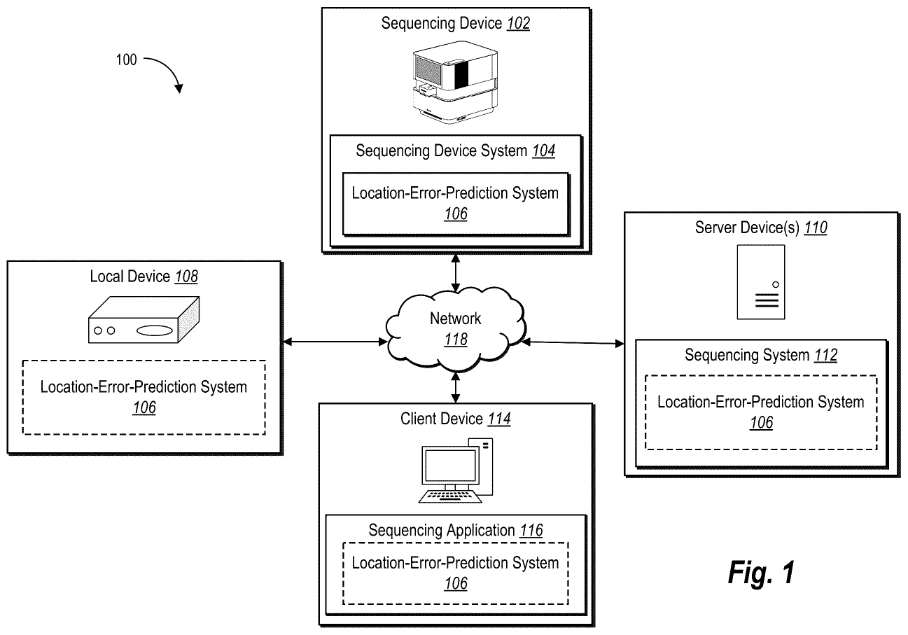

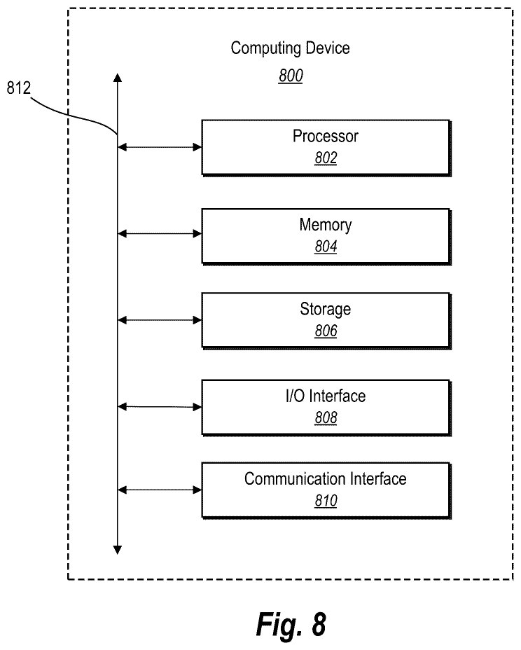

The following paragraphs describe the location-error-prediction system with respect to illustrative figures that portray example embodiments and implementations. For example, FIG. 1 illustrates a schematic diagram of a computing system 100 in which a location-error-prediction system 106 operates in accordance with one or more embodiments. As illustrated, the computing system 100 includes a sequencing device 102 connected to a local device 108 (e.g., a local server device), one or more server device(s) 110 comprising a sequencing system 112, and a client device 114. As shown in FIG. 1, the sequencing device 102, the local device 108, the server device(s) 110, and the client device 114 can communicate with each other via a network 118. The network 118 comprises any suitable network over which computing devices can communicate. Example networks are discussed in additional detail below with respect to FIG. 8. While FIG. 1 shows an embodiment of the location-error-prediction system 106, this disclosure describes alternative embodiments and configurations below.

As indicated by FIG. 1, the sequencing device 102 comprises a computing device and a sequencing device system 104 for sequencing a genomic sample or other nucleic-acid polymer. In some embodiments, by executing the sequencing device system 104 using a processor, the sequencing device 102 analyzes nucleotide fragments or oligonucleotides extracted from genomic samples to generate nucleotide reads or other data utilizing computer implemented methods and systems either directly or indirectly on the sequencing device 102. More particularly, the sequencing device 102 receives nucleotide-sample slides (e.g., flow cells) comprising nucleotide fragments (e.g., genomic DNA fragments) extracted from samples and further copies and determines the nucleobase sequence of such extracted nucleotide fragments.

In one or more embodiments, the sequencing device 102 utilizes SBS to sequence nucleotide fragments into nucleotide reads and determine nucleobase calls for the nucleotide reads. In addition or in the alternative to communicating across the network 118, in some embodiments, the sequencing device 102 bypasses the network 118 and communicates directly with the local device 108 or the client device 114. By executing the sequencing device system 104, the sequencing device 102 can further store the nucleobase calls as part of base-call data that is formatted as a binary base call (BCL) file and send the BCL file to the local device 108 and/or the server device(s) 110 comprising the sequencing system 112.

As further indicated by FIG. 1, the local device 108 is located at or near a same physical location of the sequencing device 102. Indeed, in some embodiments, the local device 108 and the sequencing device 102 are integrated into a same computing device. The local device 108 may run the location-error-prediction system 106 to generate, receive, analyze, store, and transmit digital data, such as by receiving signals of cluster of oligonucleotides and/or base-call data or determining base calls based on analyzing such signals and/or base call data. In some embodiments, the location-error-prediction system 106 operates/exists on the sequencing device 102. As indicated by the dashed lines encompassing the location-error-prediction system 106, the location-error-prediction system 106 can alternatively operate/exist on the local device 108, the server device(s) 110 within the sequencing system 112, and/or the client device 114. As shown in FIG. 1, the sequencing device 102 may send (and the local device 108 may receive) base-call data generated during a sequencing run of the sequencing device 102. By executing software in the form of the location-error-prediction system 106, the local device 108 or the sequencing system 112 may align nucleotide reads with a structural variation reference genome and determine genetic variants based on the aligned nucleotide reads.

As further indicated by FIG. 1, the server device(s) 110 are located remotely from the local device 108 and the sequencing device 102. Similar to the local device 108, in some embodiments, the server device(s) 110 include a version of the location-error-prediction system 106. Accordingly, the server device(s) 110 may generate, receive, analyze, store, and transmit digital data, such as by receiving base-call data, quality data, and other data relevant to sequencing nucleic-acid polymers. As indicated above, the sequencing device 102 may send (and the server device(s) 110 may receive) base-call data from the sequencing device 102. The server device(s) 110 may also communicate with the client device 114. In particular, the server device(s) 110 can send data to the client device 114, including read data, nucleic-acid polymer sequences, error data, and other sequencing related information.

In some embodiments, the server device(s) 110 comprise a distributed collection of servers where the server device(s) 110 include a number of server devices distributed across the network 118 and located in the same or different physical locations. Further, the server device(s) 110 can comprise a content server, an application server, a communication server, a web-hosting server, or another type of server.

As further illustrated and indicated in FIG. 1, by executing a sequencing application 116, the client device 114 can generate, store, receive, and/or send digital data. In particular, the client device 114 can receive sequencing data from the local device 108 or receive call files (e.g., BCL) and sequencing metrics from the sequencing device 102. Furthermore, the client device 114 may communicate with the local device 108 or the server device(s) 110 to receive nucleobase calls and/or other metrics, such as a base-call-quality metrics or pass-filter metrics. The client device 114 can accordingly present or display information pertaining to nucleobase calls within a graphical user interface of the sequencing application 116 to a user associated with the client device 114. For example, the client device 114 can present nucleic acid sequences, structural variant calls, and/or sequencing metrics for a sequenced genomic sample within a graphical user interface of the sequencing application 116.

Although FIG. 1 depicts the client device 114 as a desktop or laptop computer, the client device 114 may comprise various types of client devices. For example, in some embodiments, the client device 114 includes non-mobile devices, such as desktop computers or servers, or other types of client devices. In yet other embodiments, the client device 114 includes mobile devices, such as laptops, tablets, mobile telephones, or smartphones. Additional details regarding the client device 114 are discussed below with respect to FIG. 8.

As further illustrated in FIG. 1, the client device 114 includes the sequencing application 116. The sequencing application 116 may be a web application or a native application stored and executed on the client device 114 (e.g., a mobile application, desktop application). The sequencing application 116 can include instructions that (when executed) cause the client device 114 to receive data from the location-error-prediction system 106 and present, for display at the client device 114, base-call data.

As further illustrated in FIG. 1, a version of the location-error-prediction system 106 may be located and implemented (e.g., entirely or in part) on the client device 114 or the sequencing device 102. In yet other embodiments, the location-error-prediction system 106 is implemented by one or more other components of the computing system 100, such as the local device 108. In particular, the location-error-prediction system 106 can be implemented in a variety of different ways across the sequencing device 102, the local device 108, the server device(s) 110, and the client device 114. For example, the location-error-prediction system 106 can be downloaded from the server device(s) 110 to the sequencing device system 104 and/or the local device 108 where all or part of the functionality of the location-error-prediction system 106 is performed at each respective device within the computing system 100.

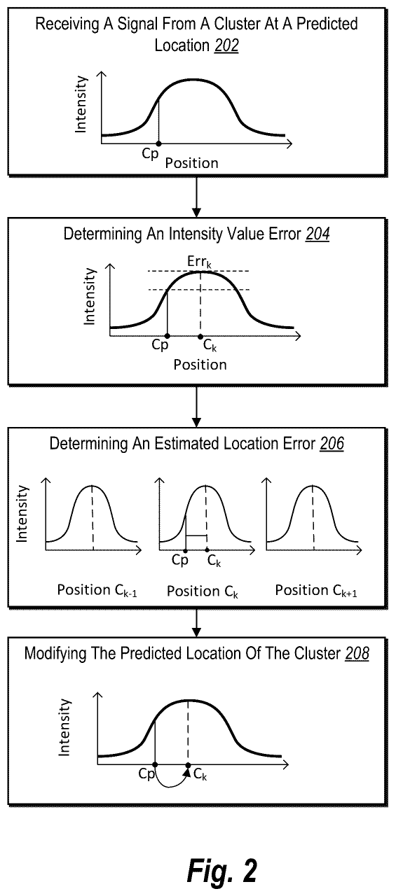

The following paragraphs provide further details concerning the location-error-prediction system 106. In accordance with one or more embodiments, FIG. 2 depicts an overview of the location-error-prediction system 106 determining an estimated location error and modifying the predicted location of a cluster of nucleotides. As shown in FIG. 2, the location-error-prediction system 106 performs a series of acts that includes an act 202 of receiving a signal from a cluster at a predicted location, an act 204 of determining an intensity value error, an act 206 of determining an estimated location error, and an act 208 of modifying the predicted location of the cluster.

As just mentioned, FIG. 2 illustrates the act 202 of receiving a signal from a cluster at a predicted location (Cₚ). In some embodiments, the location-error-prediction system 106 may receive a signal for the cluster of oligonucleotides and signals from adjacent clusters of oligonucleotides. For instance, the location-error-prediction system 106 can capture an image of the signal from the cluster of oligonucleotides and extract from the image an intensity value for signal emitted by the cluster of oligonucleotides.

In some embodiments, the location-error-prediction system 106 may detect intensity values for the cluster of nucleotides and intensity values for adjacent clusters through laser (e.g., light) excitation and imaging. Generally, during a sequencing cycle, the location-error-prediction system 106 can direct a light source with a specified wavelength at a nucleotide-sample slide (or portion of the nucleotide-sample slide) and capture an image of the clusters within the region of the nucleotide-sample slide emitting a signal. In some embodiments, the location-error-prediction system 106 captures multiple images of clusters emitting signals in different color and/or intensity channels. For instance, the location-error-prediction system 106 can capture multiple images using various channels (e.g., one image per filter or intensity channel). To illustrate, in some embodiments, the location-error-prediction system 106 utilizes a two-channel implementation by capturing two images of a region of the nucleotide-sample slide per sequencing cycle. In particular, the location-error-prediction system 106 (i) captures a first image of a tile (or other region of a nucleotide-sample slide) comprising cluster(s) emitting a signal in response to a laser in a first channel and (ii) captures a second image of the tile (or other region of a nucleotide-sample slide) comprising cluster(s) emitting another signal in response to a laser in a second channel. The first and second images can capture the intensity values of the emitted signals from the cluster and the adjacent cluster that corresponds to the channel.

As indicated above, however, the location-error-prediction system 106 can implement sequencing runs using alternative channel-based approaches. In some implementations, the location-error-prediction system 106 utilizes a four-channel implementation and captures four different images of the section of the flow cell. Similar to the two-channel implementation, the location-error-prediction system 106 can capture each image for the four-channel implementation using a different channel. Each image can capture an intensity of the emitted signal based on the channel used for that image. Thus, in some cases, each of the four images depicts the emitted signal with a different intensity. Additionally, the location-error-prediction system 106 can utilize a single channel implementation and capture one image (or a three-channel implementation and capture three images) of the section of the nucleotide-sample slide and using a specific channel to capture the intensity of the emitted signal.

In certain cases, the location-error-prediction system 106 receives a signal from a cluster at a predicted location (Cₚ) by directly receiving a fluorescent response. As described above, the fluorescent response can be a light-based indicator or fluorescent dye indicator. In certain cases, the location-error-prediction system 106 can receive the fluorescent response in the form of a fluorescent tag indicating the nucleotide base.

In contrast to an image-captured florescent response, in some embodiments, the location-error-prediction system 106 can receive a signal by receiving a voltage or electrical response from the cluster of oligonucleotides. For instance, the location-error-prediction system 106 can flow electrical currents through a DNA sample and receive changes in the electrical current and/or specific electrical signatures corresponding to the nucleotide base. For example, in some cases, the location-error-prediction system 106 can associate the variations of the electrical currents and/or electrical signatures with a specific nucleotide base (A, C, G, T). Based on the variation and or electrical signature, the location-error-prediction system 106 can identify the nucleotide base.

As further shown in FIG. 2, the location-error-prediction system 106 can perform an act 204 of determining an intensity-value error (Errₖ) for the cluster of oligonucleotides at the predicted location. As previously mentioned, the location-error-prediction system 106 determines the intensity-value error between an intensity value for the signal and an expected intensity value for the signal at the predicted location (Cₚ) within the region. As indicated above, the intensity value for the signal of the cluster is the intensity value measured by the location-error-prediction system 106 at the predicted location (Cₚ).

As indicated above, the location-error-prediction system 106 determines and uses a predicted location of a cluster of oligonucleotides. In some implementations, the location-error-prediction system 106 determines the predicted location (Cₚ) by capturing an image of the signal of the cluster and using pixels within the image to identify the predicted location (Cₚ). In certain cases, the image comprises pixels representing the signal on the nucleotide-sample slide. Based on identifying a set of pixels corresponding to the signal, the location-error-prediction system 106 predicts the location/position of the cluster by interpolating between the pixels from the set of pixels that represent the signal. The location-error-prediction system 106 can use various methods of interpolation including, but not limited to, nearest neighbor, bilinear, bicubic, lanczos, or spline. For instance, the location-error-prediction system 106 can predict a position between pixels representing the signal by interpolating between the pixels.

After the location-error-prediction system 106 measures the intensity value of the signal at the predicted location (Cₚ), the location-error-prediction system 106 can determine the expected intensity value for the signal at the predicted location (Cₚ). As mentioned above, the intensity value of the signal at the predicted location (Cₚ) indicates an illumination status (e.g., ON/OFF) of the signal in a particular channel. Based on illumination status of the signal in the particular channel, the location-error-prediction system 106 can identify the expected intensity value. In particular, the location-error-prediction system 106 can identify the average intensity value of signals with a particular illumination status. For example, based on the intensity value of the signal indicating an OFF status in a particular channel, the location-error-prediction system 106 can determine the average intensity value for all OFF signals in that particular channel. In some cases, the expected intensity value is the same across all clusters within the region of the nucleotide-sample slide in the particular channel.

As mentioned above, the location-error-prediction system 106 determines the intensity-value error between the detected or measured intensity value for the signal and the expected intensity value for the signal at the predicted location (Cₚ). In certain embodiments, the location-error-prediction system 106 can determine the intensity-value error in multiple dimensions. For example, the location-error-prediction system 106 can determine the intensity-value error in a first dimension parallel to the predicted location (e.g., along an x-axis). Relatedly, the location-error-prediction system 106 can determine the intensity-value error in a second dimension (e.g., along a y-axis). In some cases, the location-error-prediction system 106 can determine the intensity-value error in multiple channels. To illustrate, in a 2-channel implementation, the location-error-prediction system 106 can determine a first intensity-value error for a first channel and a second intensity-value error for a second channel.

As further shown in FIG. 2, the location-error-prediction system 106 performs the act 206 of determining an estimated location error for the predicted location (Cₚ). For example, the location-error-prediction system 106 determines the estimated location error for the predicted location (Cₚ) based on the intensity-value error and intensity values for other clusters within the region.

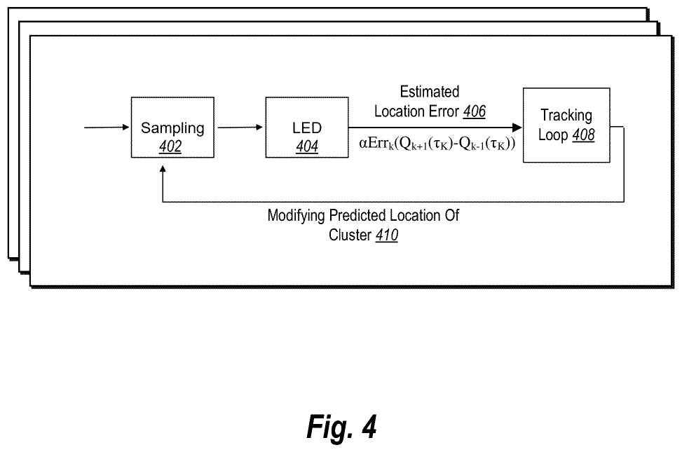

FIG. 4 and corresponding paragraphs below provide further detail about how the location-error-prediction system 106 utilizes the intensity-value error (Errₖ) of the signal, and intensity values (Qₖ₊₁ (τₖ)), (Qₖ₋₁ (τₖ)) of the of other clusters to determine the estimated location error for the predicted location (Cₚ). In certain embodiments, the location-error-prediction system 106 can determine an average estimated location error for clusters within a region on the nucleotide-sample slide. Moreover, the location-error-prediction system 106 can determine the estimated location error in multiple dimensions and/or multiple channels. For instance, the location-error-prediction system 106 can determine the estimated location error in a parallel direction (e.g., x-axis) relative to the predicted location of the cluster within the region of the nucleotide-sample slide. Additionally or alternatively, the location-error-prediction system 106 can determine the estimated location error for a particular color channel. In some cases, the location-error-prediction system 106 utilizes the estimated location error as part of a function to modify the predicted location (Cₚ) of a cluster of oligonucleotides to the corrected or modified location (Cₖ) of the cluster.

After determining the estimated location error, as further shown in FIG. 2, the location-error-prediction system 106 performs an act 208 of modifying the predicted location (Cₚ) of the cluster based on the estimated location error. Based on the modified location, the location-error-prediction system 106 can make more accurate nucleobase calls for the cluster. For example, the location-error-prediction system 106 can determine that the signal at the modified location of the cluster indicates a cytosine (C) nucleobase call, whereas the signal at the predicted location of the cluster initially indicated a guanine (G) nucleobase call. With an improved predicted location of a cluster of oligonucleotides in a given sequencing cycle, the location-error-prediction system 106 can better apply PSF compensation to a center of the cluster and better optimize or adjust an SNR for the signal to generate a more accurate adjusted signal upon which to determine the corresponding base call.

As indicated above, the location-error-prediction system 106 can modify the predicted location (Cₚ) of the cluster by repositioning the predicted location (Cₚ) of the cluster in the direction opposite to the direction indicated by the estimated location error. For example, if the estimated location error indicates that the predicted location (Cₚ) is left of the actual location (Cₖ) of the cluster, the location-error-prediction system 106 can modify the predicted location (Cₚ) by moving the predicted location (Cₚ) of the cluster to the right. In some embodiments, the location-error-prediction system 106 can modify the predicted location (Cₚ) of the cluster in multiple dimensions. For instance, the location-error-prediction system 106 can modify the predicted location (Cₚ) of the cluster in a parallel direction (e.g., along an x-axis) relative to the predicted location (Cₚ) of the cluster to correct for the estimated location error. Relatedly, the location-error-prediction system 106 can correct for the estimated location error by modifying the predicted location (Cₚ) of the cluster in a perpendicular direction (e.g., along a y-axis) relative to the predicted location. Moreover, as discussed above, the location-error-prediction system 106 can modify the predicted location of the cluster for a particular color channel.

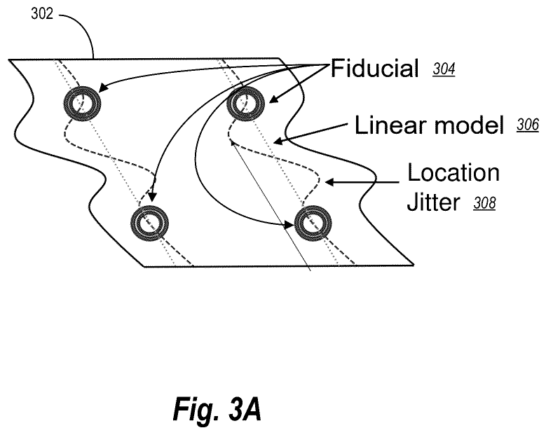

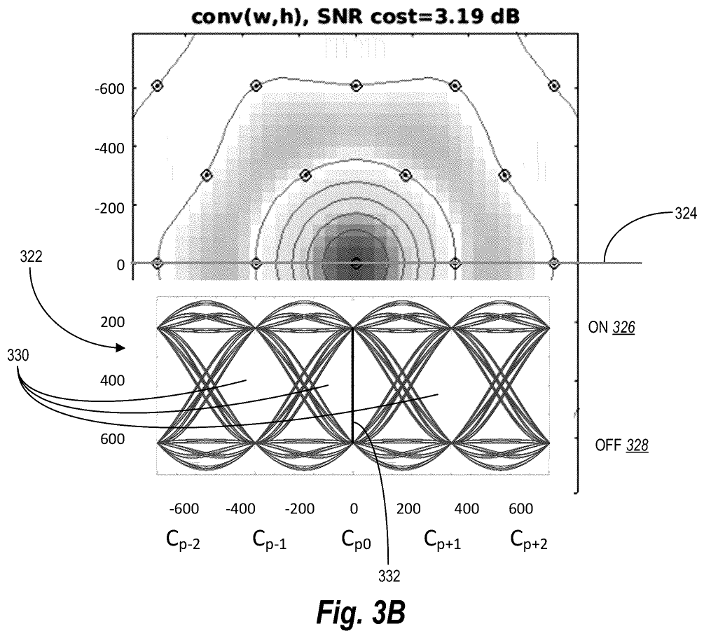

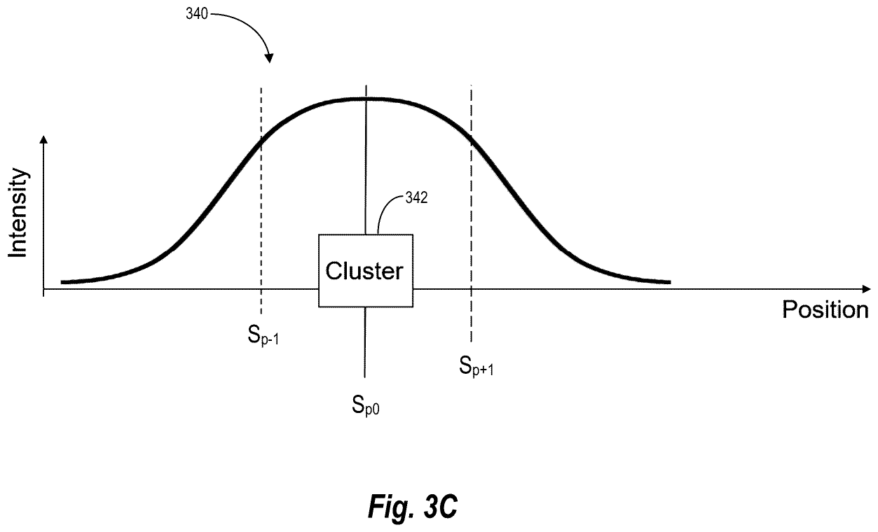

FIG. 2 provides an overview of acts performed by the location-error-prediction system 106 as part of modifying the predicted location of the cluster by utilizing the estimated location error to more closely align the predicted location of the cluster with the actual location of the cluster. In accordance with one or more embodiments, FIGS. 3A-3C illustrate diagrams of sample clusters of oligonucleotides on a nucleotide-sample slide. In particular, FIG. 3A depicts how existing sequencing systems that utilize linear models in conjunction with fiducials fail to account for location jitter while locating clusters of oligonucleotides.

As shown in FIG. 3A, a nucleotide-sample slide 302 includes fiducials 304. As discussed above, the fiducials 304 represent physical markers on the nucleotide-sample slide 302 and act as reference points for imaging of regions on the nucleotide-sample slide 302. Some existing sequencing systems establish and track the location of clusters by utilizing the fiducials 304 and a linear model 306. For instance, existing sequencing systems estimate the locations of cluster by fitting the linear model 306 to the fiducials 304. More specifically, some existing sequencing systems map the locations of the clusters based on the linear model 306 and the fiducials 304. However, as further shown in FIG. 3A, the linear model 306 cannot account for location jitter 308. In addition to jitter-tracking failures illustrated by FIG. 3A, as mentioned above, the linear model 306 fails to account for manufacturing imperfections of the nucleotide-sample slide and/or thermal expansion of the nucleotide-sample slide 302 during sequencing. Additionally, the linear model cannot fully rectify residual distortions in the image data. As used herein, the term “location jitter” refers to positional variations, deviations, and/or drift from the actual location of a cluster on the nucleotide-sample slide during sequencing. For example, movement of the nucleotide-sample slide, parts of the sequencer, and/or imaging path can cause location jitter 308. Location jitter is further described and addressed in U.S. Provisional Application No. 63/441,606, entitled, “Jitter Correction Image Analysis,” which is hereby incorporated by reference in its entirety.

As FIG. 3A shows, the location jitter 308 causes the existing sequencing system using the linear model 306 to predict incorrect locations of clusters. Because the existing sequencing system predicts incorrect locations of the clusters, the existing sequencing system also incorrectly aligns signals from such clusters with a center of the PSF and inaccurately determines SNR for the corresponding signals.

As just suggested, an existing sequencing system that determines an incorrect predicted location for a cluster of oligonucleotides tends to sample a signal from the cluster at an incorrect location for purposes of applying a PSF. In accordance with one or more embodiments, FIG. 3B depicts an ideal sampling point for a signal of a cluster of oligonucleotides in conjunction with a 1-dimensional eye diagram. In particular, FIG. 3B analogizes the quality of the response of the signal of a cluster along an x-axis 324 after equalization with the 1-dimensional eye diagram 322. Generally, the 1-dimensional eye diagram 322 indicates the quality of the signal from a given cluster along the x-axis 324. In particular, the 1-dimensional eye diagram 322 illustrates where the location-error-prediction system 106 receives the cleanest signals from the given clusters along the x-axis 324.

As illustrated in FIG. 3B, the 1-dimensional eye diagram 322 comprises an “ON” illumination indicator 326 at the top of the 1-dimensional eye diagram 322 and an “OFF” illumination indicator 328 at the bottom of the 1-dimensional eye diagram 322. As further shown in FIG. 3B, the 1-dimensional eye diagram 322 comprises an eye 330 with an eye width 332. Where the eye width 332 of the eye 330 of the 1-dimensional eye diagram is the most open (e.g., widest), the location-error-prediction system 106 receives a clean ON/OFF signal from the given cluster along the x-axis 324 because the predicted location of the cluster best matches the actual location of the cluster and the encoder of the location-error-prediction system 106 is centered for the PSF applied to the given cluster along the x-axis 324.

As further indicated by FIG. 3B, when the location-error-prediction system 106 receives the signal from a given cluster along the x-axis 324 at a predicted location that is not aligned with the actual location of the cluster along the x-axis 324, the eye 330 narrows and the location-error-prediction system 106 receives a signal with mixed inter-cluster interference. As discussed in more detail below, the location-error-prediction system 106 can account for a misaligned predicted location by modifying the predicted location of the cluster to align (or more closely align) with the actual location of the cluster.

In accordance with one or more embodiments, FIG. 3C depicts the location-error-prediction system 106 receiving (e.g., sampling) a signal from various predicted locations. In particular, FIG. 3C illustrates predicted locations with regard to a cross-section 340 of a PSF. The PSF corresponding to the cross-section 340 can describe the response of a point source (e.g., cluster of oligonucleotides). More specifically, FIG. 3C shows a cross-section 340 of a mathematically modeled PSF response of intensity values from a cluster 342. As shown in FIG. 3C, the highest intensity values occur at the peak of cross-section 340 of the PSF over the center of the cluster 342 at the actual (e.g., central) location (Sₚ₀) of the cluster and decrease as the signal from the cluster 342 moves away from the actual location (Sₚ₀) or area of the cluster 342.

As mentioned above, the location-error-prediction system 106 receives the cleanest signal from the cluster 342 when the predicted location aligns with the actual location (Sₚ₀) of the cluster 342. More specifically, the location-error-prediction system 106 receives a clear signal when the predicted location aligns with the center of the PSF. FIG. 3C illustrates an embodiment of the predicted location of the cluster 342 not aligning with the actual location (Sₚ₀) of the cluster 342.

For instance, as used herein, the term “preceding predicted location” (e.g., early sampling) (Sₚ₋₁) refers to a predicted location that comes before the actual location (Sₚ₀) of the cluster 342 in a given dimension. As shown in FIG. 3C, along a parallel dimension (e.g., x-axis), the preceding predicted location (Sₚ₋₁) can be left of the actual location (Sₚ₀) of the cluster 342. Moreover, along a perpendicular dimension (e.g., y-axis), the preceding predicted location (Sₚ₋₁) may be above the actual location (Sₚ₀) of the cluster 342. In some cases, the degree of misalignment (e.g., estimated location error) for the preceding predicted location (Sₚ₋₁) can vary. For instance, the preceding predicted location (Sₚ₋₁) can be directly before the actual location (Sₚ₀) of the cluster 342. In certain embodiments, there is a large distance between the preceding predicted location (Sₚ₋₁) and the actual location (Sₚ₀) of the cluster 342. As shown in FIG. 3C, the intensity value at the preceding predicted location (Sₚ₋₁) is lower than the intensity value at the actual location (Sₚ₀) of the cluster 342. As discussed in more detail below, the location-error-prediction system 106 can modify the preceding predicted location (Sₚ₋₁) of cluster 342 over multiple sequencing cycles.

Relatedly, the term “succeeding predicted location” (e.g., late sampling) (Sₚ₊₁) refers to a predicted location that comes after the actual location (Sₚ₀) of the cluster 342 for the given dimension. For example, along the parallel dimension (e.g., x-axis) the succeeding predicted location (Sₚ₊₁) can be right of the actual location (Sₚ₀) of the cluster 342. Relatedly, within a perpendicular dimension (e.g., along the y-axis), the succeeding predicted location (Sₚ₊₁) may occur below the actual location (Sₚ₀) of the cluster 342. In some cases, the degree of misalignment (e.g., estimated location error) for the succeeding predicted location (Sₚ₊₁) differs. For example, the succeeding predicted location (Sₚ₊₁) can immediately follow the actual location (Sₚ₀) of the cluster 342. Additionally, like the preceding predicted location (Sₚ₋₁), the intensity value at the succeeding predicted location (Sₚ₊₁) is lower than the intensity value at the actual location (Sₚ₀) of the cluster 342. As discussed above, sampling the signal at the preceding predicted location (Sₚ₋₁) or the succeeding predicted location (Sₚ₊₁) increases base calling errors. As discussed in more detail below, the location-error-prediction system 106 can modify the succeeding predicted location (Sₚ₊₁) of cluster 342 over multiple sequencing cycles.

As just discussed, the location-error-prediction system 106 can receive a signal from a cluster of oligonucleotides at a predicted location that is not aligned with the actual location of the cluster. In accordance with one or more implementations, FIG. 4 illustrates the location-error-prediction system 106 iteratively modifying a predicted location of a cluster of oligonucleotides to more closely align with a corrected location of the cluster of oligonucleotides. In particular, the location-error-prediction system 106 iteratively determines the estimated location error for the predicted location of the cluster of oligonucleotides and iteratively modifies the predicted location of the cluster based on the estimated location errors by utilizing a tracking loop.

As shown in FIG. 4, the location-error-prediction system 106 performs the act 402 of sampling the signal (e.g., as part of applying a PSF) from the cluster of oligonucleotides. In some embodiments, sampling includes capturing a signal from a cluster of oligonucleotides at a predicted location. For example, in some embodiments, the location-error-prediction system 106 samples the signal by capturing an image of a region of the nucleotide-sample slide. As suggested above, the location-error-prediction system 106 can capture, for each channel, an image of a region of a nucleotide-sample slide comprising a target cluster of oligonucleotides per sequencing cycle. In certain cases, the image comprises pixels depicting light emissions from the signal from a cluster within the region on the nucleotide-sample slide.

In one or more embodiments, when performing the act 402 of sampling the signal, the location-error-prediction system 106 detects a signal from a cluster by detecting a fluorescent response from the cluster in the captured image. In some cases, the fluorescent response can be a light-based indicator or fluorescent dye indicator. For example, the location-error-prediction system 106 can receive the fluorescent response in the form of a fluorescent tag indicating the nucleotide base. More specifically, the location-error-prediction system 106 can detect a fluorescent emission from a fluorescent tag emitting light after being excited by a light source (e.g., laser).

As indicated above, in some cases, the location-error-prediction system 106 further determines the predicted location of a cluster for sampling by (i) identifying, from the captured image, a set of pixels corresponding to a signal from a target cluster of oligonucleotides and (ii) interpolating between pixels from the set of pixels to predict a position between such pixels representing the signal from the target cluster. For instance, in some embodiments, the location-error-prediction system 106 captures an image of the signal from the cluster of oligonucleotides and identifies a set of pixels that correspond to (e.g., surround, adjoin) the signal from the cluster of oligonucleotides. In some embodiments, the center of the cluster of oligonucleotides corresponds to a single pixel. In one or more cases, the center of the cluster of oligonucleotides corresponds to multiple pixels. As indicated above, the set of pixels can represent signals emitted from the cluster of oligonucleotides.

As part of determining the predicted location, in some embodiments, the location-error-prediction system 106 determines the predicted location by identifying the locations of the fiducials on the nucleotide-sample slide (e.g., patterned nucleotide-sample slide) and interpolating between the fiducials and clusters on the nucleotide-sample slide. In certain cases, the location-error-prediction system 106 utilizes, but is not limited to, bilinear interpolation, nearest neighbor, bicubic, lanczos, or spline interpolation. In one or more instances, the location-error-prediction system 106 utilizes any combination of the aforementioned methods. In particular embodiments, the location-error-prediction system 106 can equalize the pixels before interpolation. For example, the location-error-prediction system 106 can interpolate between the set of pixels by utilizing the equalizer. Alternatively, the location-error-prediction system 106 can interpolate the pixels prior to equalization. In some embodiments, the location-error-prediction system 106 can determine the predicted location of the cluster by identifying the location (e.g., between or around interpolated pixels) with the highest intensity value because it indicates the center of the cluster (e.g., highest point of the PSF).

After capturing one or more images of a target cluster and determining the predicted location at which to sample a target cluster, in some implementations, the location-error-prediction system 106 extracts intensity values from the captured image(s) at the predicted location. In some instances, the location-error-prediction system 106 utilizes a linear equalizer to determine the intensity values for the clusters within the region by processing the captured image(s). Generally, a linear equalizer is a linear filter that can be designed or optimized to filter out noise. In certain embodiments, the equalizer can convert received dispersed-over-pixels intensity energy into the received intensity values for the cluster by linearly weighting pixel intensities. In some embodiments, the linear filter can be applied to each cluster individually, over a region on the nucleotide-sample slide, or across an entire image.

Generally, the linear equalizer (and other equalizers) reverse signal distortion during sequencing. When implemented on a sequencing device, in some embodiments, the location-error-prediction system 106 can utilize the linear equalizer to calculate the weighted sum of the intensity values of pixels that depict intensity emissions from clusters. The equalizer may be trained to produce equalizer coefficients that are configured to mix/combine intensity values of pixels that depict intensity emissions from clusters in a manner that maximizes, for example, a signal-to-noise ratio (SNR) for a given signal.

As further shown, after the location-error-prediction system 106 samples the signal from the cluster at a predicted location, the location-error-prediction system 106 can utilize a location error detector (LED) 404 to determine an estimated location error. By using the LED 404, the location-error-prediction system 106 can determine the estimated location error for the predicted location of the cluster and thereby determine whether the predicted location precedes or follows a corrected cluster location. As used herein the term, “location error detector” (or more simply LED) refers to a method, model, and/or system for detecting location errors (e.g., estimated location error) during sampling. For example, the LED 404 can determine the estimated location error between the predicted location of the cluster of oligonucleotides and the corrected location of the cluster of oligonucleotides by utilizing a stochastic gradient descent method. Generally, a stochastic gradient descent approach minimizes a loss function by iteratively adjusting the parameters of a model. Relatedly, a loss function (e.g., MSE, MAE) usually quantifies discrepancies between predicted values and actual values in a dataset. In one or more embodiments, the LED 404 can utilize the stochastic gradient descent to minimize the discrepancies between the predicted location of the cluster and the corrected location of the cluster. In some implementations, the location-error-prediction system 106 minimizes the inconsistencies between the predicted location of the cluster and the modified location of the cluster by utilizing batch least squares.

As further examples of a loss-function approach, in one or more embodiments, the location-error-prediction system 106 utilizes a mean squared error (MSE) loss function. By minimizing the MSE of the intensity-value error (e.g., between the expected intensity value and the measured intensity at the predicted location), the location-error-prediction system 106 can more closely align the predicted location of the cluster with the actual location of the cluster. In some cases, the location-error-prediction system 106 minimizes the MSE by implementing a minimum mean squared error (“MMSE”) LED 404. In one or more embodiments, the MMSE version of LED iteratively minimizes the MSE of the intensity-value error. As discussed in more detail below, the MMSE version of the LED 404 minimizes the MSE of the intensity-value error by minimizing the expected mean squared error of the intensity-value error.

As just mentioned, in some cases, the location-error-prediction system 106 utilizes an MMSE version of the LED 404 to minimize the MMSE between the expected intensity value and the measured intensity value of the cluster of oligonucleotides at the predicted location. As further shown in FIG. 4, the location-error-prediction system 106 can determine an estimated location error 406. As mentioned above, the estimated location error comprises an intensity-value error and a combination of intensity values from adjacent clusters.

In some embodiments, the location-error-prediction system 106 determines the intensity-value error by calculating the difference between the intensity value (e.g., detected or measured intensity value) for the cluster of oligonucleotides and the expected intensity value for the cluster of oligonucleotides. As discussed above, the location-error-prediction system 106 can measure the intensity value of a cluster at the predicted location by capturing an image of the cluster of oligonucleotides within the region on the nucleotide-sample slide.

In addition to measuring the intensity value for the cluster of oligonucleotides, the location-error-prediction system 106 can identify or determine the expected intensity value for the cluster of oligonucleotides in a given channel. More specifically, the location-error-prediction system 106 can use the intensity values of the cluster of oligonucleotides to determine the illumination indicators (e.g., ON/OFF status) of the cluster of oligonucleotides in the channel. Based on the illumination indicators of the cluster of oligonucleotides in the channel, the location-error-prediction system 106 can determine the expected intensity value.

As discussed above, the expected intensity value indicates a characteristic or attribute of a signal associated with the illumination indicator (e.g., ON/OFF status) of a channel and/or a nucleotide base (A, C, G, T). In particular, the expected intensity value can represent a centroid value of the probability boundaries of a nucleotide base (A, C, G, T). In some cases, the centroid value of the probability boundaries for the nucleotide base comprises an average of the intensity values that fall within the probability boundaries of the nucleotide base. For instance, the expected intensity value associated with adenine (A) is different than the expected intensity value of guanine (G). In one or more embodiments, the centroid value of the probability boundaries for the nucleotide base comprises a median value of the intensity values within the boundaries. Additionally, in certain implementations, the centroid value for the probability boundaries may be the most common (e.g., mode) intensity value within the probability boundaries for the nucleotide base.

As previously mentioned, in certain cases, the expected intensity value covers all clusters within a defined region within the nucleotide-sample slide. For example, based on the “on” status for the cluster of oligonucleotides, the expected intensity value for the cluster of oligonucleotides along a tile is the same. In some implementations, the expected intensity value can be the same across a single row or across an entire nucleotide-sample slide.

As indicated above, the location-error-prediction system 106 aims to minimize the intensity-value error (e.g., minimize the MSE of the intensity-value error). In one or more implementations the location-error-prediction system 106 minimizes the intensity-value error by minimizing the expected mean squared error (EMSE) of the intensity-value error. In some embodiments, the EMSE of the intensity-value error can be modeled as E[(Errₖ)²] where Errₖ represents the intensity-value error. As described above, the intensity-value error (Errₖ) can comprise the expected intensity value and the measured intensity value of the signal of the cluster at the predicted location. In some embodiments, the intensity-value error (Errₖ) can be modeled as (Qₖ(τₖ)−Aₖ), where (Qₖ(τₖ)) represents the measured intensity value for the signal of the cluster of oligonucleotides given the predicted location (τₖ), (k) represents the cluster index (e.g., an index sequence that identifies a particular cluster or well), and (Aₖ) represents the expected intensity value for the signal of the cluster of oligonucleotides in the given dimension. As discussed above, in some cases, (Aₖ) can represent the expected intensity value by mapping the illumination (e.g., ON/OFF) status of the signal to the expected intensity value in the specified dimension. For instance, in some embodiments, the signal of the cluster is not illuminated (e.g., off signal) in a first intensity channel in perpendicular direction relative to the predicted location of the cluster. Based on the “off” signal, the location-error-prediction system 106 can determine (e.g., map to) the expected intensity value for the signal of the cluster in the given dimension. For instance, the location-error-prediction system 106 can map the “off” signal to an average intensity value for “off” signals.

Having a model for EMSE, in certain implementations, the location-error-prediction system 106 can minimize the EMSE of the intensity-value error E[(Q_{c}(τₖ)−A_{c})²] by taking the derivative of the EMSE between the expected intensity value and the measured intensity value for the signal modeled as: (E[(Qₖ(τₖ)−Aₖ)²]). In some implementations, the derivative of the intensity-value error with respect to predicted location (τₖ) can be further modeled as:

$$( \frac{\partial E [ {( {Err}_{k} )}^{2} ]}{\partial {τ}_{k}} ) .$$

In one or more implementations, the derivative of the intensity-value error can be modeled as:

$$E [ \frac{\partial [ {( {Err}_{k} )}^{2} ]}{\partial {τ}_{k}} ] ,$$

which can become

$$E [ 2 {Err}_{k} \cdot \frac{\partial {Err}_{k}}{\partial {τ}_{k}} ] .$$

In certain cases, since Errₖ=Qₖ(τₖ)−Aₖ, the location-error-prediction system 106 can substitute Errₖ with (Qₖ (τₖ)−Aₖ). Thus, in certain models, the derivative of the intensity-value error

$$( \frac{\partial {Err}_{k}}{\partial {τ}_{k}} )$$

can be represented as

$$\frac{\partial {Q}_{k} ( {τ}_{k} )}{\partial {τ}_{k}} - \frac{\partial {A}_{k}}{\partial {τ}_{k}} .$$

The location-error-prediction system 106 can take the derivative of

$$( \frac{\partial {Q}_{k} ( {τ}_{k} )}{\partial {τ}_{k}} - \frac{\partial {A}_{k}}{\partial {τ}_{k}} )$$

resulting in

$$( \frac{\partial {Q}_{k} ( {τ}_{k} )}{\partial {τ}_{k}} - 0 ) .$$

Therefore, according to certain models, the location-error-prediction system 106 can model the derivative of the intensity-value error as

$$2 {Err}_{k} \cdot \frac{\partial {Q}_{k} ( {τ}_{k} )}{\partial {τ}_{k}} .$$

As suggested above, the location-error-prediction system 106 can represent the minimized EMSE of the intensity-value error as

$$2 {Err}_{k} . \frac{\partial {Q}_{k} ( {τ}_{k} )}{\partial {τ}_{k}} .$$

However, based on the available information, in certain implementations deriving the intensity value error for the signal from the cluster of oligonucleotides given the predicted location (Qₖ (τₖ)) is difficult. Therefore, in certain cases, the location-error-prediction system 106 can approximate ∂Qₖ(τₖ) by utilizing the intensity values from neighboring clusters or neighboring pixels. In particular, the location-error-prediction system 106 can approximate ∂Qₖ(τₖ) by combining the intensity values of neighboring clusters. For example, the location-error-prediction system 106 can determine the difference between the measured intensity values of a succeeding cluster Qₖ₊₁(τₖ) and a preceding cluster Qₖ₋₁ (τₖ) along the given dimension. Thus, the derivative of the intensity value error for the signal from the cluster of oligonucleotides given the predicted location can be modeled as:

$$\frac{\partial {Q}_{k} ( {τ}_{k} )}{\partial {τ}_{k}} = {Q}_{k + 1} ( {τ}_{k} ) - {Q}_{k - 1} ( {τ}_{k} ) .$$

Therefore, in a dimension perpendicular to (e.g., along a y-axis) the predicted location, the location-error-prediction system 106 can approximate ∂Qₖ(τₖ) by determining the difference between the intensity values of the preceding cluster Qₖ₋₁ (τₖ) (e.g., beneath the target cluster) and a succeeding cluster Qₖ₊₁(τₖ) (e.g., above the target cluster).

As previously indicated, the location-error-prediction system 106 can determine an estimated location error based on combining the intensity-value error and the intensity values for other clusters within the region. As indicated above, the location-error-prediction system 106 can represent the estimated location error 406 according to the following model: αErrₖ (Qₖ₊₁(τₖ)−Qₖ₋₁ (τK)). The step-size constant (a) represents the learning rate of the LED 404 for the given dimension. In some cases, the step-size (e.g., learning rate) helps determine the degree of correction or change for the estimated location error. In certain embodiments, the location-error-prediction system 106 can utilize an adaptive learning rate that adjusts the learning rate based on historical information from the location-error-prediction system 106. For example, the location-error-prediction system 106 can determine the step-size for the next stochastic gradient decent by utilizing AdaGrad, AdaDelta, RMSProp, and/or Adam. In certain implementations, the step-size a may be a fixed learning rate where a remains constant for every iteration during sequencing.

As further shown in the model, the estimated location error 406 can include the intensity-value error (Errₖ). As discussed above, the intensity-value error represents the difference between the detected or measured intensity value of the signal and the expected intensity value of the signal for the cluster.

As FIG. 4 illustrates, the estimated location error 406 can include the combined intensity values of signals from adjacent clusters (Qₖ₊₁(τₖ)−Qₖ₋₁(τₖ)) in the given dimension. For instance, in certain cases, the location-error-prediction system 106 can measure the intensity values of clusters in a perpendicular dimension in relation to the predicted location of the cluster. In certain cases, the location-error-prediction system 106 can take the difference between the first intensity value of a first adjacent cluster (Qₖ₊₁(τₖ)) and a second intensity value of a second adjacent cluster (Qₖ₋₁(τₖ)) in a perpendicular direction relative to the predicted location of the cluster. To illustrate, the location-error-prediction system 106 can find the difference between the first intensity value of a first adjacent cluster (Qₖ₊₁(τₖ)) below the predicted location of the cluster and the second intensity value of a second adjacent cluster (Qₖ₋₁(τₖ)) above the predicted location of the cluster in the perpendicular direction relative to the predicted location of the cluster. As discussed above, the location-error-prediction system 106 can determine the difference between the intensity values of the first adjacent cluster and the second adjacent cluster in various dimensions. For instance, the location-error-prediction system 106 can combine the intensity values of adjacent clusters in a parallel direction relative to the predicted location of the cluster.

As further shown in FIG. 4, the location-error-prediction system 106 utilizes a tracking loop 408. Generally, the tracking loop 408 monitors the estimated location error 406. For example, in some cases, the tracking loop 408 iteratively determines the estimated location error 406 for the predicted location of the cluster and modifies the predicted location to more closely align with a corrected location of the cluster. For example, the location-error-prediction system 106 updates the estimated location error gradient to minimize the distance between the predicted location and the corrected location of the cluster.

As just indicated above the location-error-prediction system 106 can modify the predicted location of the cluster based on tracking the estimated location error. In particular, the tracking loop enables the location-error-prediction system 106 to iteratively determine the estimated location error after each modification and modify the predicted location of the cluster towards the actual location of the cluster. In some embodiments, as the tracking loop converges towards a minimum value between the expected intensity value and the measured intensity at the predicted location, location-error-prediction system 106 can maintain that convergence by tracking the intensity-value error over time and not diverging from the minima.

Unlike many existing sequencing systems, in certain embodiments, the location-error-prediction system 106 can utilize the tracking loop 408 to modify the predicted location of a cluster in real time. For instance, the location-error-prediction system 106 can determine how quickly the tracking loop 408 determines the estimated location error and modifies the predicted location of the cluster. In particular, the location-error-prediction system 106 can determine the estimated location error for the cluster and, based on the estimated location error of the cluster, the location-error-prediction system 106 can adjust the predicted location for the subsequent cluster. For example, if the estimated location error indicates that the predicted location of the cluster comes after (e.g., to the right of) the actual location of the cluster, the location-error-prediction system 106 can modify the predicted location of the subsequent cluster by adjusting the predicted location of the cluster to the left. By leveraging the knowledge of the current cluster to more closely align the predicted location of the cluster with the actual location of the cluster, the location-error-prediction system 106 can quickly and accurately identify the actual location of the cluster without adding extra fiducials.

In addition to an initial loop, the location-error-prediction system 106 can utilize multiple loops to align the predicted location of the cluster with the actual location of the cluster. For example, the location-error-prediction system 106 can implement a first tracking loop in a parallel dimension relative to the predicted location of the cluster while utilizing a second tracking loop in a perpendicular dimension relative to the predicted location of the cluster. In certain embodiments, the first tracking loop only modifies (e.g., updates) the predicted location of the cluster in the parallel dimension. Likewise, in certain cases, the second tracking loop modifies the predicted location of the cluster in the perpendicular dimension relative to the predicted location of the cluster. In certain implementations, as the location-error-prediction system 106 modifies the predicted location of the cluster in both dimensions, the predicted location of the cluster converges towards the actual location of the cluster.

At a same or subsequent sequencing cycle, FIG. 4 further shows the location-error-prediction system 106 modifying the predicted location of the cluster 410. In particular, the location-error-prediction system 106 modifies the predicted location of the cluster 410 based on the estimated location error 406. For example, based on the estimated location error 406, the location-error-prediction system 106 modifies the predicted location of the cluster in an opposite direction than the direction indicated by the estimated location error 406. For example, if the estimated location error 406 indicates that the predicted location of the cluster is above the actual location of the cluster, the location-error-prediction system 106 modifies the predicted location of the cluster down to more closely align with the actual location of the cluster. As discussed in more detail below, the location-error-prediction system 106 can modify the predicted location of the cluster in multiple dimensions, channels, and across various sequencing cycles.

In addition or in the alternative to the embodiments described above with respect to FIG. 4, the location-error-prediction system 106 can perform a different approach to a loop for tracking and modifying a predicted location of a cluster. In contrast to an image-captured florescent response, in certain implementations, the location-error-prediction system 106 detects the signal by detecting a voltage or electrical response from the cluster of oligonucleotides. As mentioned above, in some embodiments, the location-error-prediction system 106 can flow electrical currents and/or charges through a DNA sample. In some cases, changes in the electrical current may indicate a specific nucleotide base (A, C, G, T). For example, a decrease in the electrical current may indicate Adenine. As another illustration, an increase in the electrical current may signal Guanine and/or specific electrical signatures corresponding to the nucleotide base. In some cases, the location-error-prediction system 106 can associate the variations of the electrical currents and/or electrical signatures with a specific nucleotide base (A, C, G, T). Based on the variation and or electrical signature, the location-error-prediction system 106 can identify the nucleotide base.

As discussed above, the location-error-prediction system 106 can determine the expected intensity value for a cluster based on the base call. In some embodiments, the location-error-prediction system 106 can determine the expected intensity value based on the illumination indicator (e.g., ON/OFF status) of a cluster in a channel. For example, in certain cases, based on the nucleobase call, the location-error-prediction system 106 can determine the illumination indicator of the cluster in the given channel. Based on the illumination indicator, the location-error-prediction system 106 can find an expected intensity value associated with the illumination status of the channel. More specifically, the location-error-prediction system 106 may determine the centroid value for a given illumination status of the signal within the given channel. For example, the centroid value may be the average of intensity values for signals that are off (e.g., unilluminated) for the given channel. As mentioned above, an off signal may still illicit an intensity value. In some embodiments, the centroid value for the illumination status of the cluster of oligonucleotides for the given channel may be the median intensity value or most common intensity value for all off signals in the given channel. In certain implementations, the location-error-prediction system 106 can determine the expected intensity value in multiple channels.

As discussed in FIG. 4, the location-error-prediction system 106 can determine the intensity-value error based on the intensity values of adjacent clusters. In contrast to using adjacent clusters as the basis for an intensity-value difference, in some embodiments, the location-error-prediction system 106 determines the intensity-value difference (and the estimated location error 406) based on pixels representing intensity values associated with a target cluster. As indicated above, the location-error-prediction system 106 can receive a signal by receiving a captured image of the signal of the target cluster. In certain cases, the captured image comprises pixels, where the pixels correspond to the PSF of the signal of the target cluster. In certain implementations, the signal of the target cluster at the predicted location spans more than one pixel in a given dimension. In such embodiments, the location-error-prediction system 106 can extract intensity values from the pixels corresponding to the signal of the target cluster and intensity values from pixels adjacent to the target cluster. For example, the location-error-prediction system 106 can utilize an intensity extraction algorithm or other image processing method that measures the brightness of pixels representing the signal of the target cluster. For instance, the intensity extraction algorithm can define an integration area and extract intensity values within the integration area. In some cases, the location-error-prediction system 106 can define an integration area covering multiple pixels corresponding to the PSF of the signal. In certain implementations, the location-error-prediction system 106 can extract a first intensity value for a first pixel adjacent to the target cluster and a second intensity value for a second pixel adjacent to the target cluster within the integration area.

As an illustration of pixel-based approach to determining the intensity-value difference, the location-error-prediction system 106 can determine the estimated location error 406 by utilizing the intensity values for other locations represented by pixels adjacent to the predicted location of the cluster. In particular, the location-error-prediction system 106 can determine an adjacent-pixels intensity-value difference between pixels adjacent to the predicted location of the cluster. For example, the location-error-prediction system 106 can determine the adjacent-pixels intensity-value difference between a first intensity value for a first pixel positioned adjacent to the predicted location of the cluster and a second intensity value for a second pixel positioned adjacent to the predicted location of the cluster. To illustrate, the location-error-prediction system 106 can subtract the first intensity value for the first pixel adjacent to the predicted location of the cluster from the second intensity value for the second pixel positioned adjacent to the predicted location of the cluster.

In some embodiments, the location-error-prediction system 106 can determine the estimated location error 406 by combining the intensity-value error with the adjacent-pixels intensity value difference. Based on the estimated location error, the location-error-prediction system 106 can modify the predicted location of the cluster. Such a modified predicted location can come from a cluster-specific estimated location error for a single cluster of oligonucleotides and for a single sequencing cycle (e.g., current or subsequent sequencing cycle).

In addition or in the alternative to determining a cluster-specific estimated location error, in some embodiments, the location-error-prediction system 106 can determine the estimated location error 406 as a multi-cluster estimated location error for multiple clusters. For instance, the location-error-prediction system 106 can determine an average estimated location error as the estimated location error 406 for multiple clusters within a defined region for a given dimension. In some implementations, the location-error-prediction system 106 can receive signals from clusters within a tile of the nucleotide-sample slide by receiving an image of the signals within the tile. As discussed above, based on the imaged signals, the location-error-prediction system 106 can determine the intensity-values of the signals from the pixels making up the image. The location-error-prediction system 106 can further determine the intensity value error between the measured intensity values of the signals and the expected intensity values of the signals within the tile for the particular dimension (e.g., along the x-axis). Moreover, the location-error-prediction system 106 can determine the difference between the intensity values of neighboring clusters for all clusters within the tile along the particular dimension. Based on the intensity-value errors from multiple clusters and the combined intensity values from adjacent clusters, the location-error-prediction system 106 can determine estimated location errors for all clusters within the region. In some cases, the location-error-prediction system 106 can determine an average estimated location error for a given row and/or column of clusters.

In addition or in the alternative to determining a cycle-specific estimated location error, the location-error-prediction system 106 can determine a cross-cycle estimated location error over multiple sequencing cycles. For instance, the location-error-prediction system 106 can determine the estimated location error 406 for a nucleotide-sample slide for a first sequencing cycle. In some cases, the location-error-prediction system 106 can determine the estimated location error 406 for subsequent sequencing cycles. Based on averaging the estimated location error 406 for a defined number of sequencing cycles (e.g., 20 sequencing cycles), the location-error-prediction system 106 can modify the predicted location of clusters on the nucleotide-sample slide for the 20 cycles. In certain implementations, the location-error-prediction system 106 only modifies the predicted locations of clusters in the first sequencing cycle.

In some embodiments, the location-error-prediction system 106 can determine the estimated location error by utilizing the symmetric PSF response of the signal from the cluster. In particular, the location-error-prediction system 106 can determine the estimated location error based on pixels depicting the PSF of the cluster.

As just mentioned, the location-error-prediction system 106 can determine the estimated location error by utilizing the symmetry of the PSF for the signal. As discussed above, the PSF can describe the response of a point source. In some embodiments, the PSF is a Gaussian PSF that represents the intensity values of the signal over a bell-shaped distribution. For instance, the peak of the distribution represents the highest intensity value at the center of the cluster, and the intensity values of the PSF symmetrically decrease as the distance increases from the center of the distribution. To illustrate, where pixels depict the measured intensity values of the PSF, the pixel(s) at the center of the PSF have the highest intensity values and the intensity values of the pixels decrease along a normal distribution as the pixels increase in distance away from the center of the PSF.

As previously mentioned, if the predicted location of the cluster is aligned with the actual location of the cluster, the location-error-prediction system 106 samples the signal at the center of the PSF. In such cases, due to the symmetric nature of the PSF, the measured intensity value of a first pixel preceding the cluster at the predicted location and the intensity value of a second pixel following the cluster is the same and the estimated location error is zero. However, if the predicted location is not the actual location of the cluster, the location-error-prediction system 106 samples the signal along the distribution of the PSF. In particular, the location-error-prediction system 106 measures the intensity value of a pixel that is more highly correlated with one side of the PSF. In an embodiment, where the signal of the cluster is more highly correlated with one side of the PSF, the intensity values of the first pixel preceding the cluster and of the second pixel following the cluster differ. Based on the correlation at the predicted location, the location-error-prediction system 106 can modify the predicted location of the cluster to equalize the correlation between the first and second pixel.

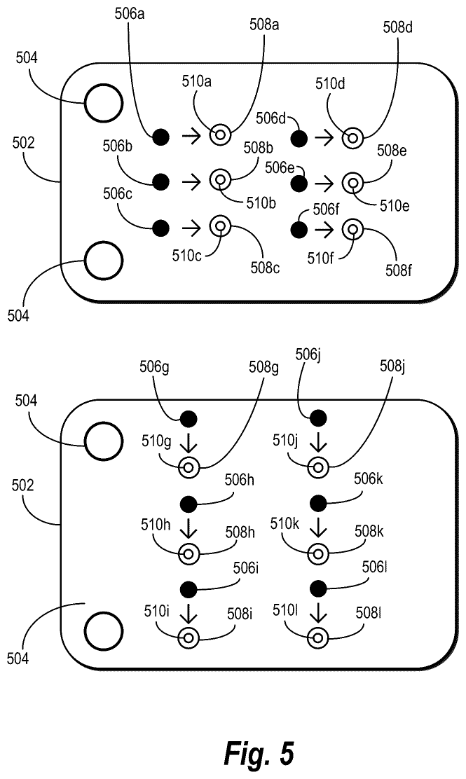

As described above, the location-error-prediction system 106 can modify a predicted location of a cluster of oligonucleotides to generate a corrected location. In accordance with one or more embodiments, FIG. 5 illustrates the location-error-prediction system 106 modifying predicted locations (e.g., preceding predicted locations and/or succeeding predicted locations) of different clusters of oligonucleotides based on estimated location errors. In particular, as described below, the location-error-prediction system 106 modifies predicted locations 506a, 506b, 506c, 506d, 506e, and 506f of clusters 510a, 510b, 510c, 510d, 510e, and 510f, respectively, to corrected locations 508a, 508b, 508c, 508d, 508e, and 508f, respectively, in a first dimension (e.g., parallel dimension relative to a nucleotide-sample-slide axis). Additionally or alternatively, the location-error-prediction system 106 modifies predicted locations 506g, 506h, 506i, 506j, 506k, and 506l of clusters 510g, 510h, 510i, 510j, 510k, and 5101, respectively, to corrected locations 508g, 508h, 508l, 508j, 508k, and 508l, respectively, in a second dimension (e.g., perpendicular dimension relative to a nucleotide-sample-slide axis). In some embodiments, as depicted in FIG. 5, the clusters 510a-510f are the same oligonucleotide clusters as the clusters 510g-5101 within a region of a nucleotide-sample slide 502 (e.g., the same clusters from images in different channels). By contrast, in some embodiments, the clusters 510a-510f are different oligonucleotide clusters from the clusters 510g-5101 (e.g., different clusters within different nucleotide-sample-slide regions). The following description applies to both such scenarios in the alternative. For purposes of explaining predicted and corrected locations relative to different dimension, however, FIG. 5 uses different reference numbers for the clusters 510a-510f and the clusters 510g-5101.

As mentioned above, in some embodiments, the location-error-prediction system 106 modifies the predicted locations 506a-506f of the clusters 510a-510f, respectively, by modifying coordinates (x and/or y coordinates) for the predicted locations 506a-506f in the direction opposite to the gradient indicated by estimated location error. For instance, as just mentioned and as shown in FIG. 5, the location-error-prediction system 106 can modify the predicted location 506a of the cluster 510a in one or more dimensions. FIG. 5 illustrates the location-error-prediction system 106 modifying the predicted location 506a of the cluster 510a to more closely align with the corrected location 508a of the cluster 510a on the nucleotide-sample slide 502 comprising fiducials 504. As shown in FIG. 5, the predicted location 506a of the cluster 510a is not aligned with the corrected location 508a of the cluster 510a along a parallel dimension relative to an axis of the nucleotide-sample slide 502 (e.g., along the x-axis). In particular, the predicted location 506a of the cluster 510a precedes the corrected location 508a of the cluster 510a along the first dimension. Because the estimated location error indicates that the predicted location 506a is left of (e.g., precedes) the corrected location 508a of the cluster 510a, the location-error-prediction system 106 modifies the predicted location 506a of the cluster 510a to the right (e.g., toward a succeeding location).

As further shown in FIG. 5, the location-error-prediction system 106 can modify multiple predicted locations, such as by modifying one or more of the predicted locations 506a-506f of the clusters 510a-510f, respectively, to the corrected locations 508a-508f of the clusters 510a-510f, respectively. For example, the location-error-prediction system 106 can modify the predicted locations 506a-506f of the clusters 510a-510f within a defined region based on the average estimated location error for the region. As discussed above, the location-error-prediction system 106 can determine the estimated location errors for the clusters 510a-510f within a tile, sub-tile, or other region of the nucleotide-sample slide 502. The location-error-prediction system 106 can determine an average estimated location error by averaging the estimated location errors of the predicted locations 506a-506f within the tile, sub-tile, or other region of the nucleotide-sample slide 502. Based on the average estimated location error, the location-error-prediction system 106 can modify the predicted locations 506a-506f of the clusters 510a-510f within the tile, sub-tile, or other region. Additionally or alternatively, in one or more implementations, the location-error-prediction system 106 can determine the average estimated location error for a single row (e.g., within a region of the nucleotide-sample slide 502) and modify predicted locations of clusters of oligonucleotides (e.g., the predicted locations 506a and 506d of the clusters 510a and 510d) within the row based on the average estimated location error for the clusters of oligonucleotides within the row. In some embodiments, the location-error-prediction system 106 does not need to perform iterative updates of the modified predicted locations for the clusters within the defined region. For example, in some cases, if the location-error-prediction system 106 calibrates a sequencing device (e.g., determines the estimated location error and updates the predicted location of the clusters based on prior knowledge and/or measurements of the sequencing device 102), the location-error-prediction system 106 can modify the predicted locations of the clusters of oligonucleotides by determining the average estimated location error for the clusters within the region and performing a non-iterative batch update based on the average estimated location error.

In some embodiments, the location-error-prediction system 106 can determine the average estimated location error for a defined region and modify the predicted locations 506a-506l of the clusters outside of the defined region. For example, the location-error-prediction system 106 can determine the average estimated location error for predicted locations of oligonucleotide clusters within a tile, sub-tile, or other region of the nucleotide-sample slide 502 and modify the predicted locations of the oligonucleotide clusters (e.g., the clusters 510a-510f) for the tile, sub-tile, or other region within the nucleotide-sample slide 502. Alternatively, the location-error-prediction system 106 can determine the average estimated location error for predicted locations of oligonucleotide clusters within the entirety of the nucleotide-sample slide 502 and modify the predicted locations of the oligonucleotide clusters for the entirety of the nucleotide-sample slide 502. In certain implementations, the location-error-prediction system 106 can modify one or more of the predicted locations 506a-506l of one or more of the clusters 510a-5101, respectively, within a sub-region of the defined region. For example, the location-error-prediction system 106 can determine the average estimated location error for a region of the nucleotide-sample slide 502 and modify a single row of predicted locations (e.g., a row including the predicted locations 506a and 506d) within the region. Furthermore, in one or more cases, the location-error-prediction system 106 can define the region for determining the average estimated location error. For example, based on the average estimated location error for the region, the location-error-prediction system 106 can expand or minimize the region. Alternatively, the location-error-prediction system 106 can receive user input defining the relevant region of the nucleotide-sample slide 502.

In addition to one-dimensional location adjustments, as further shown in FIG. 5, the location-error-prediction system 106 can modify a predicted location of a cluster in a second dimension. For example, the location-error-prediction system 106 can modify the predicted location 506g of the cluster 510g in a perpendicular dimension relative to an axis of the nucleotide-sample slide 502 (e.g., along the y-axis). As FIG. 5 illustrates, the location-error-prediction system 106 can adjust the predicted location 506g of the cluster 510g downward towards the corrected location 508g of the cluster 510g. As discussed above, the location-error-prediction system 106 can determine the average estimated location error for the clusters 510g, 510h, 510i, 510j, 510k, and 5101 over a region of the nucleotide-sample slide 502 in a given dimension (e.g., along the y-axis) and modify one or more of the predicted locations 506g, 506h, 506i, 506j, 506k, and 506l of one or more of the clusters 510g, 510h, 510i, 510j, 510k, and 5101, respectively, in that dimension. Such modified predicted locations are shown in FIG. 5, for instance, as the corrected locations 508g-508l for the clusters 510g-5101, respectively. In some embodiments, the region for a set of clusters (e.g., a set from the clusters 510g-5101) may differ in the given dimension. For example, the location-error-prediction system 106 can modify the predicted locations 506g-506l of the clusters 510g-5101 in a first region of the nucleotide-sample slide 502 along a perpendicular dimension (e.g., along an x-axis of the nucleotide-sample slide 502) and modify the predicted locations 506a-506f of the clusters 510a-510f along a parallel dimension (e.g., a y-axis of the nucleotide-sample slide 502) in a second region of the nucleotide-sample slide 502 (e.g., smaller or larger than the first region).

As indicated above, in some embodiments, the location-error-prediction system 106 can modify one or more of the predicted locations 506a-506l based on the average estimated location error over a number of sequencing cycles. For example, the location-error-prediction system 106 can determine the estimated location errors for the clusters 510a-5101 within the region the nucleotide-sample slide 502 for a sequencing cycle in a given dimension. The location-error-prediction system 106 can further track the estimated location error for the clusters 510a-5101 over multiple sequencing cycles and determine the average estimated location error for the clusters 510a-5101 at the predicted location 506a-5101 over the multiple sequencing cycles. In some embodiments, the location-error-prediction system 106 can modify the predicted locations 506a-506l of the clusters 510a-5101 for the current sequencing cycle or for subsequent sequencing cycles. For example, the location-error-prediction system 106 can determine the average estimated location error for a defined region of the nucleotide-sample slide 502 in a given dimension over 20 sequencing cycles.

Based on the average estimated location error, the location-error-prediction system 106 can modify the predicted locations 506a-506l in the opposite direction indicated by the direction of the average estimated location error. For example, if the average estimated location error after a subset of sequencing cycles (e.g., 20, 30, 50 sequencing cycles) for a row of clusters at predicted locations indicates that the predicted locations are positioned after (e.g., to the right of) corresponding corrected locations along a dimension relative to an axis of the nucleotide-sample slide 502, for purposes of the subset of sequencing cycles (e.g., for base calling), the location-error-prediction system 106 modifies the predicted locations of the clusters within the row to a position before (e.g., to the left of) the corresponding corrected locations. Such a row may include, for example, a row including the clusters 510a and 510d at the predicted locations 506a and 506d corresponding to the corrected locations 508a and 508d, respectively. In some embodiments, based on the average estimated location error for the subset of sequencing cycles (e.g., 50 sequencing cycles), the location-error-prediction system 106 modifies the predicted locations of the clusters within the row for a smaller subset of the sequencing cycles (e.g., an initial 20 sequencing cycles).

As indicated above, the location-error-prediction system 106 can determine the average estimated location error for clusters at a preceding predicted location within a defined region of a nucleotide-sample slide in the given dimension over multiple sequencing cycles. Based on the average estimated location error, the location-error-prediction system 106 can modify the preceding predicted locations of the clusters to more closely align with the actual location of the clusters. Relatedly, the location-error-prediction system 106 can determine the average estimated location error for clusters at a succeeding predicted location with the defined region in the given dimension over multiple sequencing cycles. The location-error-prediction system 106 can further modify the succeeding predicted location to align with the actual location of the cluster based on the average estimated location error.

As mentioned above, in one or more embodiments, the location-error-prediction system 106 utilizes a nucleotide-sample slide (e.g., the nucleotide-sample slide 502) comprising clusters of oligonucleotides arranged in a hexagonal layout. In such implementations, the dimensions between adjacent clusters arranged in a hexagonal layout within the nucleotide-sample slide comprises three axes of symmetry. In cases where the nucleotide-sample slide comprises oligonucleotide clusters in such a hexagonal layout, the location-error-prediction system 106 can convert the three axes of symmetry into a separate x-axis and y-axis. As discussed above, the location-error-prediction system 106 can further determine the estimated location error for one or more predicted locations of oligonucleotide clusters along such a converted x-axis and converted y-axis.

As mentioned above, the location-error-prediction system 106 can modify the predicted location of the clusters for one or more channels. As discussed above, in some embodiments, the location-error-prediction system 106 receives an image of a PSF response in a given channel. In some cases, the PSF response in different color channels varies due to the different wavelengths associated with the color channel. For example, the PSF response of the signal from the cluster in a green channel differs from the PSF response of the signal from the cluster in a red or blue channel. Because of the differences in the PSF response, in certain implementations, the location-error-prediction system 106 can determine the estimated location error for a particular color channel. Based on the estimated location error for the particular color channel, the location-error-prediction system 106 can modify the predicted location of the cluster in the particular color channel. For example, based on a two-channel implementation, the location-error-prediction system 106 can determine, for a first color channel, a first estimated location error for the predicted location of the cluster within a region of the nucleotide-sample slide. Moreover, the location-error-prediction system 106 can determine, for a second color channel, a second estimated location error for the predicted location of the cluster within the region of the nucleotide-sample slide. Based on the first estimated location error for the first color channel, the location-error-prediction system 106 can modify the predicted location of the cluster for purposes of the first color channel (e.g., for identifying a modified predicted location of the cluster in pixel(s) of a first-color-channel image from which intensity values are extracted for base calling the cluster). Similarly, based on the second estimated location error for the second color channel, the location-error-prediction system 106 can modify the predicted location of the cluster for purposes of the second color channel (e.g., for identifying a modified predicted location of the cluster in pixel(s) of a second-color-channel image from which intensity values are extracted for base calling the cluster). In a four-channel implementation, the location-error-prediction system 106 can determine the estimated location error for each of the four color channels and modify the predicted location of the cluster within each color channel. The location-error-prediction system 106 may likewise determine the estimated location error for three-channel and single channel implementations.

As described above, in a two-channel implementation, the location-error-prediction system 106 independently modifies the predicted location of the cluster in the first color channel and the predicted location of the cluster in the second color channel. In particular, the location-error-prediction system 106 can determine the estimated location error for the predicted location of the cluster within the particular color channel. For example, as described above, the location-error-prediction system 106 can determine the intensity-value error between the intensity value for the signal in a particular color channel and the expected intensity value for the signal in the particular color channel at the predicted location within the region. Moreover, the location-error-prediction system 106 can determine the estimated location error for the predicted location of the cluster based on the intensity-value error in the particular color channel and the intensity values for other clusters in the particular color channel. For example, the location-error-prediction system 106 can determine the difference between a first intensity value of a first adjacent cluster in a first color channel and a second intensity value of a second adjacent cluster in the first color channel. Based on the intensity-value error and difference between the first intensity value of the first adjacent cluster and the second intensity value of the second adjacent cluster in the first color channel, the location-error-prediction system 106 can determine the estimated location error for the first channel. After the system determines the estimated location error for the first color channel, the location-error-prediction system 106 can modify the predicted location of the cluster in the first color channel. Moreover, the location-error-prediction system 106 can perform the same steps and determine the estimated location error for the predicted location of the cluster in a second color channel.

In certain cases, after the location-error-prediction system 106 modifies a predicted location of a cluster of oligonucleotides, the location-error-prediction system 106 can redetermine a base call for the cluster at the current sequencing cycle. More specifically, based on the signal of the cluster of oligonucleotides, the location-error-prediction system 106 can determine a base call for the cluster of oligonucleotides at the predicted location at the current sequencing cycle. In certain implementations, after modifying the predicted location of the cluster of oligonucleotides, the location-error-prediction system 106 can redetermine the base call for the cluster of oligonucleotides based on the signal from the cluster at the modified predicted location. For example, in an embodiment where the location-error-prediction system 106 initially made a guanine base call for a cluster of oligonucleotides at a predicted location, the location-error-prediction system 106 can redetermine the base call. More specifically, after modifying the predicted location, the location-error-prediction system 106 can redetermine that the base call for the cluster of oligonucleotides is adenine. Likewise, the location-error-prediction system 106 can determine (e.g., reprocess) one or more subsequent base calls for the cluster in one or more subsequent sequencing cycles. More specifically, based on a subsequent signal from the cluster of oligonucleotides at the modified predicted location, the location-error-prediction system 106 can determine a subsequent base call for the cluster of oligonucleotides at a subsequent sequencing cycle.

To be clear, in some embodiments, the location-error-prediction system 106 does not redetermine the base call of a cluster of oligonucleotides (or base calls for multiple clusters) based on a modified predicted location. In such embodiments, the location-error-prediction system 106 can use the modified predicted location of the cluster to detect a signal more accurately from (and determine base calls more accurately for) the cluster in one or more subsequent sequencing cycles. Regardless of whether initial base calls or redetermined base calls are used, in one or more implementations, the location-error-prediction system 106 can generate a binary base call (BCL) file with information about the base call and modified predicted location of the cluster. For instance, the location-error-prediction system 106 can generate the BCL file showing the redetermined base call and its corresponding modified predicted location (e.g., in coordinates).

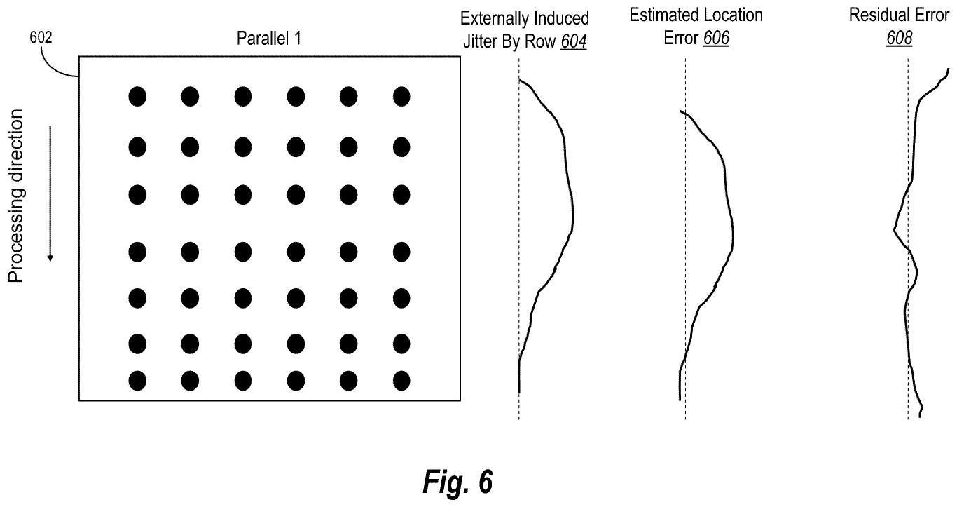

As discussed above, the location-error-prediction system 106 can determine the estimated location error and modify the predicted location of the cluster to more accurately align with a corrected location of the cluster. FIG. 6 illustrates improved cluster location tracking by utilizing the disclosed location-error-prediction system with a tracking loop in accordance with one or more implementations. As discussed above, jitter, thermal expansion, and/or other dynamic movement of the nucleotide-sample slide 602 causes the system to receive a signal that is not aligned with the center of the PSF emitted from the cluster. More specifically, in certain cases, jitter misaligns the predicted location of the cluster with the actual location of the cluster.

As shown in FIG. 6, the externally induced jitter by row 604 causes the predicted location of the cluster to deviate in an image of a nucleotide-sample-slide region from the actual location of the cluster. As further shown in FIG. 6, the estimated location error 606, which measures the error between the predicted location and corrected location of the cluster, begins to follow the externally induced jitter by row 604 as the tracking loop iteratively modifies the predicted location of the cluster. Based on the estimated location error 606 following the externally induced jitter by row 604, the location-error-prediction system 106 can modify the predicted location of the cluster so that the predicted location is more closely aligned with the actual location of the cluster.

As illustrated in FIG. 6, the residual error 608 (e.g., the residual location offset) shows the degree of misalignment between the predicted location of the cluster and the corrected location of the cluster after the location-error-prediction system 106 modifies the predicted location of the cluster. As shown in FIG. 6, the residual error 608 at the beginning of the processing is high and decreases as the estimated location error 606 follows the externally induced jitter by row 604. FIG. 6 further shows the residual error 608 converging to more closely align the predicted location of the cluster with the actual location of the cluster. In particular, FIG. 6 shows that as the location-error-prediction system 106 utilizes the tracking loop, the residual error between the predicted location and the actual location of the cluster decreases. As discussed above, the location-error-prediction system 106 can monitor the estimated location error 606, the location-error-prediction system 106 can dynamically track and align the predicted location of the cluster with the actual location of the cluster by monitoring the residual error over time and preventing the residual error from diverging.

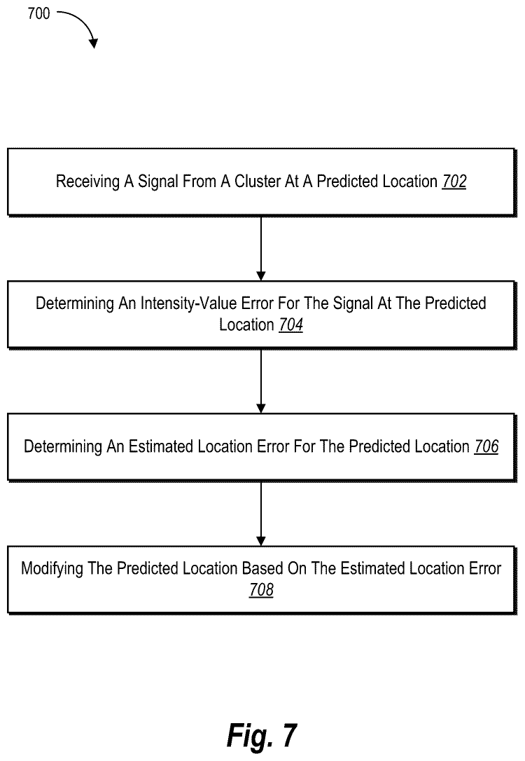

FIGS. 1-6, the corresponding text and the examples provide a number of different methods, systems, devices, and non-transitory computer-readable media of the location-error-prediction system 106. In addition to the foregoing, one or more embodiments can also be described in terms of flowcharts comprising acts for accomplishing particular results, as shown in FIG. 7. FIG. 7 may be performed with more or fewer acts. Further, the acts may be performed in different orders. Additionally, the acts described herein may be repeated or performed in parallel with one another or in parallel with different instances of the same or similar acts.

FIG. 7 illustrates a flowchart of a series of acts 700 for determining an estimated location error for the predicted location in accordance with one or more embodiments. While FIG. 7 illustrates acts according to one embodiment, alternative embodiments may omit, add to, reorder, and/or modify any of the acts shown in FIG. 7. In some implementations, the acts of FIG. 7 are performed as part of a method. In some instances, a non-transitory computer-readable medium stores instructions thereon that, when executed by at least one processor, cause a computing device to perform the acts of FIG. 7. In some implementations, a system performs the acts of FIG. 7. For example, in one or more cases, a system includes at least one processor and a non-transitory computer readable medium comprising instructions that, when executed by the at least one processor, cause the system to perform the acts of FIG. 7.

As shown in FIG. 7, the series of acts 700 includes an act 702 of receiving a signal from a cluster at a predicted location, an act 704 of determining an intensity-value error for the signal at the predicted location, an act 706 of determining an estimated location error for the predicted location, and an act 708 of modifying the predicted location based on the estimated location error. For example, the series of acts 700 can include acts to perform any of the operations described in the following clauses:

CLAUSE 1. A computer-implemented method comprising:
receiving, for a current sequencing cycle, a signal from a cluster of oligonucleotides at a predicted location of the cluster of oligonucleotides within a region of a nucleotide-sample slide;
determining an intensity-value error between an intensity value for the signal and an expected intensity value for the signal at the predicted location within the region;
determining, based on the intensity-value error and intensity values for other locations within the region, an estimated location error for the predicted location of the cluster of oligonucleotides; and
modifying, based on the estimated location error, the predicted location of the cluster of oligonucleotides for at least a sequencing cycle.

CLAUSE 2. The computer-implemented method of clause 1, wherein at least the sequencing cycle comprises the current sequencing cycle or a subsequent sequencing cycle.

CLAUSE 3. The computer-implemented method of clause 1, further comprising:
determining a base call for the cluster of oligonucleotides at the current sequencing cycle based on the signal from the cluster of oligonucleotides at the predicted location; and
redetermining the base call for the cluster of oligonucleotides at the current sequencing cycle based on the signal from the cluster of oligonucleotides at the modified predicted location.

CLAUSE 4. The computer-implemented method of clause 1, further comprising determining a subsequent base call for the cluster of oligonucleotides at a subsequent sequencing cycle based on a subsequent signal from the cluster of oligonucleotides at the modified predicted location.

CLAUSE 5. The computer-implemented method of clause 1, further comprising:
iteratively determining estimated location errors for the predicted location of the cluster of oligonucleotides across a subset of sequencing cycles; and
modifying, based on the iteratively determined estimated location errors, the predicted location of the cluster of oligonucleotides for at least the sequencing cycle after the subset of sequencing cycles.

CLAUSE 6. The computer-implemented method of clause 1, further comprising:
receiving, for a subsequent sequencing cycle, a subsequent signal from the cluster of oligonucleotides at the predicted location within the region;
determining, based on an additional intensity-value error for the subsequent signal and additional intensity values for the other locations within the region, an additional estimated location error; and
modifying, based on the estimated location error and the additional estimated location error, the modified predicted location of the cluster of oligonucleotides.

CLAUSE 7. The computer-implemented method of clause 1, wherein determining the estimated location error based on the intensity values for the other locations comprising other clusters of oligonucleotides within the region comprises:
determining an adjacent-clusters intensity-value difference between a first intensity value of a first adjacent cluster of oligonucleotides positioned adjacent to the cluster of oligonucleotides and a second intensity value of a second adjacent cluster of oligonucleotides positioned adjacent to the cluster of oligonucleotides; and
combining the intensity-value error of the cluster of oligonucleotides with the adjacent-clusters intensity-value difference to determine the estimated location error for the predicted location.

CLAUSE 8. The computer-implemented method of clause 1, wherein determining the estimated location error based on the intensity values for the other locations represented by pixels positioned adjacent to the predicted location of the cluster of oligonucleotides comprises:
determining an adjacent-pixels intensity-value difference between a first intensity value for a first pixel positioned adjacent to the predicted location of the cluster of oligonucleotides and a second intensity value for a second pixel positioned adjacent to the predicted location of the cluster of oligonucleotides; and
combining the intensity-value error of the cluster of oligonucleotides with the adjacent-pixels intensity-value difference to determine the estimated location error for the predicted location.

CLAUSE 9. The computer-implemented method of clause 1, further comprising:
receiving the signal from the cluster of oligonucleotides and signals from one or more other clusters of oligonucleotides within the region;
determining, based on one or more intensity-value errors for the one or more other clusters of oligonucleotides and the intensity values for the other locations comprising other clusters of oligonucleotides within the region, one or more additional estimated locations errors for one or more predicted locations of the one or more other clusters of oligonucleotides;
determining, based on the estimated location error for the predicted location and the one or more additional estimated locations errors for the one or more predicted locations, an average estimated location error; and
modifying the predicted location of the cluster of oligonucleotides based on the average estimated location error.

CLAUSE 10. The computer-implemented method of clause 1, further comprising:
determining the estimated location error in a parallel direction relative to the predicted location of the cluster of oligonucleotides within the region of the nucleotide-sample slide; and
modifying the predicted location of the cluster of oligonucleotides in the parallel direction to correct for the estimated location error.

CLAUSE 11. The computer-implemented method of clause 1, further comprising:
determining the estimated location error in a perpendicular direction relative to the predicted location of the cluster of oligonucleotides within the region of the nucleotide-sample slide; and
modifying the predicted location of the cluster of oligonucleotides in the perpendicular direction to correct for the estimated location error.

CLAUSE 12. The computer-implemented method of clause 1, further comprising:
determining the intensity-value error between the intensity value for the signal in a particular channel and the expected intensity value for the signal in the particular channel at the predicted location within the region; and
determining, based on the intensity-value error in the particular channel and the intensity values for the other locations comprising other clusters of oligonucleotides in the particular channel, the estimated location error for the predicted location of the cluster of oligonucleotides.

CLAUSE 13. The computer-implemented method of clause 1, wherein modifying the predicted location of the cluster of oligonucleotides comprises adjusting the predicted location of the cluster of oligonucleotides in an opposite direction than a direction indicated by the estimated location error.

CLAUSE 14. The computer-implemented method of clause 1, wherein receiving the signal from the cluster of oligonucleotides at the predicted location comprises capturing, for the current sequencing cycle, an image of the signal from the cluster of oligonucleotides at the predicted location within the region of the nucleotide-sample slide.

CLAUSE 15. The computer-implemented method of clause 1, wherein determining the predicted location of the cluster of oligonucleotides within the region comprises:
identifying, from a captured image, a set of pixels corresponding to the signal from the cluster of oligonucleotides; and
interpolating between pixels from the set of pixels to predict a position between pixels representing the signal from the cluster of oligonucleotides.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Implementations in which images are obtained in different color channels, for example, coinciding with different labels used to distinguish one nucleobase type from another are particularly applicable. In some implementations, the process to determine the nucleotide sequence of a target nucleic acid (i.e., a nucleic-acid polymer) can be an automated process. Preferred implementations include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as the release of pyrophosphate; or the like. In implementations, where two or more different nucleotides are present in a sequencing reagent, the different nucleotides can be distinguishable from each other, or alternatively, the two or more different labels can be indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred implementations include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242 (1), 84-9; Ronaghi, M. (2001) “Pyrosequencing sheds light on DNA sequencing.” Genome Res. 11 (1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to the incorporation of nucleotides at the features of the array. An image can be obtained after the array is treated with a particular nucleotide type (e.g., A, T, C, or G). Images obtained after the addition of each nucleotide type will differ with regard to which features in the array are detected. These differences in the image reflect the different sequence content of the features on the array. However, the relative locations of each feature will remain unchanged in the images. The images can be stored, processed, and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.) and is also described in WO 91/06678 and WO 07/123,744, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing implementations, the labels do not substantially inhibit extension under SBS reaction conditions. However, the detection labels can be removable, for example, by cleavage or degradation. Images can be captured following the incorporation of labels into arrayed nucleic acid features. In particular implementations, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such implementations, each image will show nucleic acid features that have incorporated nucleotides of a particular type. Different features are present or absent in the different images due to the different sequence content of each feature. However, the relative position of the features will remain unchanged in the images. Images obtained from such reversible terminator-SBS methods can be stored, processed, and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular implementations, some or all of the nucleotide monomers can include reversible terminators. In such implementations, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30-second exposure to long-wavelength UV light. Thus, either disulfide reduction or photocleavage can be used as a cleavable linker. Another approach to reversible termination is the use of natural termination that ensues after the placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100800, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some implementations can utilize the detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes an apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on the presence of their respective signals and incorporation of the fourth nucleotide type into the nucleic acid can be determined based on the absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary implementation that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some implementations can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due to the different sequence content of each feature, but the relative position of the features will remain unchanged in the images. Images obtained from ligation-based sequencing methods can be stored, processed, and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some implementations can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such implementations, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed, and analyzed as set forth herein. In particular, the data can be treated as an image in accordance with the exemplary treatment of optical images and other images that is set forth herein.

Some implementations can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed, and analyzed as set forth herein.

Some SBS implementations include the detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular implementations, different target nucleic acids can be treated in a common reaction vessel or on a surface of a particular substrate. This allows convenient delivery of sequencing reagents, removal of unreacted reagents, and detection of incorporation events in a multiplex manner. In implementations using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle, or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for the detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing implementation as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, a “sample” (and its derivatives) is used in its broadest sense and includes any specimen, culture, and the like that is suspected of including a target. In some implementations, the sample comprises DNA, RNA, PNA, LNA, chimeric or hybrid forms of nucleic acids. The sample can include any biological, clinical, surgical, agricultural, atmospheric, or aquatic-based specimen containing one or more nucleic acids. The term also includes any isolated nucleic acid sample such a genomic DNA, fresh-frozen, or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample, and normal tissue sample, or sample from a single source that contains two distinct forms of genetic material such as maternal and fetal DNA obtained from a maternal subject, or the presence of contaminating bacterial DNA in a sample that contains plant or animal DNA. In some implementations, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another implementation, low molecular weight material includes enzymatically or mechanically fragmented DNA. The sample can include cell-free circulating DNA. In some implementations, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some implementations, the sample can be an epidemiological, agricultural, forensic, or pathogenic sample. In some implementations, the sample can include nucleic acid molecules obtained from an animal such as a human or mammalian source. In another implementation, the sample can include nucleic acid molecules obtained from a non-mammalian source such as a plant, bacteria, virus, or fungus. In some implementations, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one implementation, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example, derived from a buccal swab, paper, fabric, or other substrates that may be impregnated with saliva, blood, or other bodily fluids. As such, in some implementations, the nucleic acid sample may comprise low amounts of, or fragmented portions of DNA, such as genomic DNA. In some implementations, target sequences can be present in one or more bodily fluids including but not limited to, blood, sputum, plasma, semen, urine, and serum. In some implementations, target sequences can be obtained from hair, skin, tissue samples, autopsy or remains of a victim. In some implementations, nucleic acids including one or more target sequences can be obtained from a deceased animal or human. In some implementations, target sequences can include nucleic acids obtained from non-human DNA such a microbial, plant, or entomological DNA. In some implementations, target sequences or amplified target sequences are directed to purposes of human identification. In some implementations, the disclosure relates generally to methods for identifying characteristics of a forensic sample. In some implementations, the disclosure relates generally to human identification methods using one or more target-specific primers disclosed herein or one or more target-specific primers designed using the primer design criteria outlined herein. In one implementation, a forensic or human identification sample containing at least one target sequence can be amplified using any one or more of the target-specific primers disclosed herein or using the primer criteria outlined herein.

The components of the location-error-prediction system 106 can include software, hardware, or both. For example, the components of the location-error-prediction system 106 can include one or more instructions stored on a computer-readable storage medium and executable by processors of one or more computing devices (e.g., the client device 114). When executed by the one or more processors, the computer-executable instructions of the location-error-prediction system 106 can cause the computing devices to perform the structural variant detection methods described herein. Alternatively, the components of the location-error-prediction system 106 can comprise hardware, such as special-purpose processing devices to perform a certain function or group of functions. Additionally, or in the alternative, the components of the location-error-prediction system 106 can include a combination of computer-executable instructions and hardware.

Furthermore, the components of the location-error-prediction system 106 performing the functions described herein with respect to the location-error-prediction system 106 may, for example, be implemented as part of a stand-alone application, as a module of an application, as a plug-in for applications, as a library function or functions that may be called by other applications, and/or as a cloud-computing model. Thus, components of the location-error-prediction system 106 may be implemented as part of a stand-alone application on a personal computing device or a mobile device. Additionally, or alternatively, the components of the location-error-prediction system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Implementations of the present disclosure may comprise or utilize a special purpose or general-purpose computer including computer hardware, such as, for example, one or more processors and system memory, as discussed in greater detail below. Implementations within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general-purpose or special-purpose computer system. Computer-readable media that store computer-executable instructions are non-transitory computer-readable storage media (devices). Computer-readable media that carry computer-executable instructions are transmission media. Thus, by way of example, and not limitation, implementations of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid-state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links that can be used to carry desired program code means in the form of computer-executable instructions or data structures and which can be accessed by a general-purpose or special-purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some implementations, computer-executable instructions are executed on a general-purpose computer to turn the general-purpose computer into a special-purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (Saas), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 8 illustrates a block diagram of a computing device 800 that may be configured to perform one or more of the processes described above. One will appreciate that one or more computing devices such as the computing device 800 may implement the location-error-prediction system 106 and the sequencing device system 104. As shown by FIG. 8, the computing device 800 can comprise a processor 802, a memory 804, a storage device 806, an I/O interface 1008808 and a communication interface 810, which may be communicatively coupled by way of a communication infrastructure 812. In certain implementations, the computing device 800 can include fewer or more components than those shown in FIG. 8. The following paragraphs describe components of the computing device 800 shown in FIG. 8 in additional detail.

In one or more implementations, the processor 802 includes hardware for executing instructions, such as those making up a computer program. As an example, and not by way of limitation, to execute instructions for dynamically modifying workflows, the processor 802 may retrieve (or fetch) the instructions from an internal register, an internal cache, the memory 804, or the storage device 806 and decode and execute them. The memory 804 may be a volatile or non-volatile memory used for storing data, metadata, and programs for execution by the processor(s). The storage device 806 includes storage, such as a hard disk, flash disk drive, or another digital storage device, for storing data or instructions for performing the methods described herein.

The I/O interface 808 allows a user to provide input to, receive output from, and otherwise transfer data to and receive data from computing device 800. The I/O interface 808 may include a mouse, a keypad or a keyboard, a touch screen, a camera, an optical scanner, network interface, modem, other known I/O devices, or a combination of such I/O interfaces. The I/O interface 808 may include one or more devices for presenting output to a user, including, but not limited to, a graphics engine, a display (e.g., a display screen), one or more output drivers (e.g., display drivers), one or more audio speakers, and one or more audio drivers. In certain implementations, the I/O interface 808 is configured to provide graphical data to a display for presentation to a user. The graphical data may be representative of one or more graphical user interfaces and/or any other graphical content as may serve a particular implementation.

The communication interface 810 can include hardware, software, or both. In any event, the communication interface 810 can provide one or more interfaces for communication (such as, for example, packet-based communication) between the computing device 800 and one or more other computing devices or networks. As an example, and not by way of limitation, the communication interface 810 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 810 may facilitate communications with various types of wired or wireless networks. The communication interface 810 may also facilitate communications using various communication protocols. The communication infrastructure 812 may also include hardware, software, or both that couples components of the computing device 800 to each other. For example, the communication interface 810 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary implementations thereof. Various implementations and aspects of the present disclosure(s) are described with reference to details discussed herein, and the accompanying drawings illustrate the various implementations. The description above and drawings are illustrative of the disclosure and are not to be construed as limiting the disclosure. Numerous specific details are described to provide a thorough understanding of various implementations of the present disclosure.

The present disclosure may be embodied in other specific forms without departing from its spirit or essential characteristics. The described implementations are to be considered in all respects only as illustrative and not restrictive. For example, the methods described herein may be performed with fewer or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer readable medium comprising instructions that, when executed by the at least one processor, cause the system to:
receive, for a current sequencing cycle, a signal from a cluster of oligonucleotides at a predicted location of the cluster of oligonucleotides within a region of a nucleotide-sample slide;
determine an intensity-value error between an intensity value for the signal and an expected intensity value for the signal at the predicted location within the region;
determine, based on the intensity-value error and intensity values for other locations within the region, an estimated location error for the predicted location of the cluster of oligonucleotides; and
modify, based on the estimated location error, the predicted location of the cluster of oligonucleotides for at least a sequencing cycle.

2. The system of claim 1, wherein at least the sequencing cycle comprises the current sequencing cycle or a subsequent sequencing cycle.

3. The system of claim 1, further comprising instructions that, when executed by the at least one processor cause the system to:
determine a base call for the cluster of oligonucleotides at the current sequencing cycle based on the signal from the cluster of oligonucleotides at the predicted location; and
redetermine the base call for the cluster of oligonucleotides at the current sequencing cycle based on the signal from the cluster of oligonucleotides at the modified predicted location.

4. The system of claim 1, further comprising instructions that, when executed by the at least one processor cause the system to determine a subsequent base call for the cluster of oligonucleotides at a subsequent sequencing cycle based on a subsequent signal from the cluster of oligonucleotides at the modified predicted location.

5. The system of claim 1, further comprising instructions that, when executed by the at least one processor cause the system to:
iteratively determine estimated location errors for the predicted location of the cluster of oligonucleotides across a subset of sequencing cycles; and
modify, based on the iteratively determined estimated location errors, the predicted location of the cluster of oligonucleotides for at least the sequencing cycle after the subset of sequencing cycles.

6. The system of claim 1, further comprising instructions that, when executed by the at least one processor, cause the system to:
receive, for a subsequent sequencing cycle, a subsequent signal from the cluster of oligonucleotides at the predicted location within the region;
determine, based on an additional intensity-value error for the subsequent signal and additional intensity values for the other locations within the region, an additional estimated location error; and
modify, based on the estimated location error and the additional estimated location error, the modified predicted location of the cluster of oligonucleotides.

7. The system of claim 1, further comprising instructions that, when executed by the at least one processor, cause the system to determine the estimated location error based on the intensity values for the other locations comprising other clusters of oligonucleotides within the region by:
determining an adjacent-clusters intensity-value difference between a first intensity value of a first adjacent cluster of oligonucleotides positioned adjacent to the cluster of oligonucleotides and a second intensity value of a second adjacent cluster of oligonucleotides positioned adjacent to the cluster of oligonucleotides; and
combining the intensity-value error of the cluster of oligonucleotides with the adjacent-clusters intensity-value difference to determine the estimated location error for the predicted location.

8. The system of claim 1, further comprising instructions that, when executed by the at least one processor, cause the system to determine the estimated location error based on the intensity values for the other locations represented by pixels positioned adjacent to the predicted location of the cluster of oligonucleotides by:
determining an adjacent-pixels intensity-value difference between a first intensity value for a first pixel positioned adjacent to the predicted location of the cluster of oligonucleotides and a second intensity value for a second pixel positioned adjacent to the predicted location of the cluster of oligonucleotides; and
combining the intensity-value error of the cluster of oligonucleotides with the adjacent-pixels intensity-value difference to determine the estimated location error for the predicted location.

9. The system of claim 1, further comprising instructions that, when executed by the at least one processor, cause the system to:
receive the signal from the cluster of oligonucleotides and signals from one or more other clusters of oligonucleotides within the region;
determine, based on one or more intensity-value errors for the one or more other clusters of oligonucleotides and the intensity values for the other locations comprising other clusters of oligonucleotides within the region, one or more additional estimated locations errors for one or more predicted locations of the one or more other clusters of oligonucleotides;
determine, based on the estimated location error for the predicted location and the one or more additional estimated locations errors for the one or more predicted locations, an average estimated location error; and
modify the predicted location of the cluster of oligonucleotides based on the average estimated location error.

10. The system of claim 1, further comprising instructions that, when executed by the at least one processor, cause the system to:
determine the estimated location error in a parallel direction relative to the predicted location of the cluster of oligonucleotides within the region of the nucleotide-sample slide; and
modify the predicted location of the cluster of oligonucleotides in the parallel direction to correct for the estimated location error.

11. A non-transitory computer readable medium storing instructions that, when executed by at least one processor, cause a system to:
receive, for a current sequencing cycle, a signal from a cluster of oligonucleotides at a predicted location of the cluster of oligonucleotides within a region of a nucleotide-sample slide;
determine an intensity-value error between an intensity value for the signal and an expected intensity value for the signal at the predicted location within the region;
determine, based on the intensity-value error and intensity values for other locations within the region, an estimated location error for the predicted location of the cluster of oligonucleotides; and
modify, based on the estimated location error, the predicted location of the cluster of oligonucleotides for at least a sequencing cycle.

12. The non-transitory computer readable medium of claim 11, further storing instructions that, when executed by the at least one processor, cause the system to:
determine the estimated location error in a perpendicular direction relative to the predicted location of the cluster of oligonucleotides within the region of the nucleotide-sample slide; and
modify the predicted location of the cluster of oligonucleotides in the perpendicular direction to correct for the estimated location error.

13. The non-transitory computer readable medium of claim 11, further storing instructions that, when executed by the at least one processor, cause the system to:
determine the intensity-value error between the intensity value for the signal in a particular channel and the expected intensity value for the signal in the particular channel at the predicted location within the region; and
determine, based on the intensity-value error in the particular channel and the intensity values for the other locations comprising other clusters of oligonucleotides in the particular channel, the estimated location error for the predicted location of the cluster of oligonucleotides.

14. The non-transitory computer readable medium of claim 11, further storing instructions that, when executed by the at least one processor cause the system to modify the predicted location of the cluster of oligonucleotides by adjusting the predicted location of the cluster of oligonucleotides in an opposite direction than a direction indicated by the estimated location error.

15. The non-transitory computer readable medium of claim 11, further comprising instructions that, when executed by the at least one processor cause the system to receive the signal from the cluster of oligonucleotides at the predicted location by capturing, for the current sequencing cycle, an image of the signal from the cluster of oligonucleotides at the predicted location within the region of the nucleotide-sample slide.

16. A computer-implemented method comprising:
receiving, for a current sequencing cycle, a signal from a cluster of oligonucleotides at a predicted location of the cluster of oligonucleotides within a region of a nucleotide-sample slide;
determining an intensity-value error between an intensity value for the signal and an expected intensity value for the signal at the predicted location within the region;
determining, based on the intensity-value error and intensity values for other locations within the region, an estimated location error for the predicted location of the cluster of oligonucleotides; and
modifying, based on the estimated location error, the predicted location of the cluster of oligonucleotides for at least a sequencing cycle.

17. The computer-implemented method of claim 16, wherein at least the sequencing cycle comprises the current sequencing cycle or a subsequent sequencing cycle.

18. The computer-implemented method of claim 16, further comprising:
determining a base call for the cluster of oligonucleotides at the current sequencing cycle based on the signal from the cluster of oligonucleotides at the predicted location; and
redetermining the base call for the cluster of oligonucleotides at the current sequencing cycle based on the signal from the cluster of oligonucleotides at the modified predicted location.

19. The computer-implemented method of claim 16, further comprising:
iteratively determining estimated location errors for the predicted location of the cluster of oligonucleotides across a subset of sequencing cycles; and
modifying, based on the iteratively determined estimated location errors, the predicted location of the cluster of oligonucleotides for at least the sequencing cycle after the subset of sequencing cycles.

20. The computer-implemented method of claim 16, wherein determining the predicted location of the cluster of oligonucleotides within the region comprises:
identifying, from a captured image, a set of pixels corresponding to the signal from the cluster of oligonucleotides; and
interpolating between pixels from the set of pixels to predict a position between pixels representing the signal from the cluster of oligonucleotides.
